(19) United States
(12) Patent Application Publication
(10) Pub. No.: US 2023/0255105 A1
(43) Pub. Date: Aug. 10, 2023

(54) ORGANIC COMPOUNDS WITH DELAYED FLUORESCENCE EMISSION AND CIRCULARLY POLARISED LUMINESCENCE AND USE THEREOF

(71) Applicants: COMMISSARIAT A L'ENERGIE ATOMIQUE ET AUX ENERGIES ALTERNATIVES, Paris (FR); CENTRE NATIONAL DE LA RECHERCHE SCIENTIFIQUE, Paris (FR); UNIVERSITÉ PARIS-SACLAY, Gif-sur-Yvette (FR); UNIVERSITÉ DE RENNES 1, Rennes (FR); ECOLE NATIONALE SUPÉRIEURE DE CHIMIE DE RENNES, Rennes (FR); INSTITUT NATIONAL DES SCIENCES APPLIQUEES DE RENNES, Rennes (FR)

(72) Inventors: Grégory Pieters, Guyancourt (FR); Ludovic Favereau, Montauban de Bretagne (FR); Laurelie Poulard, Aulnay-Sous-Bois (FR); Max Coehlo, Orsay (FR); Lucas Frederic, Le Plessis-Pâté (FR); Sitthichok Kasemthaveechok, Rennes (FR); Jeanne Crassous, Thorigné-Fouillard (FR)

(73) Assignees: COMMISSARIAT A L'ENERGIE ATOMIQUE ET AUX ENERGIES ALTERNATIVES, Paris (FR); CENTRE NATIONAL DE LA RECHERCHE SCIENTIFIQUE, Paris (FR); UNIVERSITÉ PARIS-SACLAY, Gif-sur-Yvette (FR); UNIVERSITÉ DE RENNES 1, Rennes (FR); ECOLE NATIONALE SUPÉRIEURE DE CHIMIE DE RENNES, Rennes (FR); INSTITUT NATIONAL DES SCIENCES APPLIQUEES DE RENNES, Rennes (FR)

(21) Appl. No.: 18/015,563

(22) PCT Filed: Jul. 9, 2021
(86) PCT No.: PCT/EP2021/069232

§ 371 Date: Jan. 11, 2023

(30) Foreign Application Priority Data
Jul. 16, 2020 (FR) 2007467

(51) Int. Cl. H10K 85/60; C07D 491/22; H10K 50/11

(52) U.S. Cl. CPC H10K 85/657; C07D 491/22; H10K 50/11; H10K 2101/20

(57) ABSTRACT

The present invention relates to compounds simultaneously having thermally activated delayed fluorescence (TADF), circularly polarised luminescence (CPL) and aggregation-induced emission enhancement (AIEE) properties. The invention also relates to the use of such compounds as a photocatalyst or as a dopant, in particular in the emitting layers of light-emitting diodes (OLEDs), as well as light-emitting devices or light-emitting diodes (OLEDs) comprising such compounds.

### TECHNICAL FIELD OF THE INVENTION

The present invention relates to compounds simultaneously having thermally activated delayed fluorescence (TADF), circularly polarised luminescence (CPL) and aggregation-induced emission enhancement (AIEE).

The invention also relates to the use of such compounds as a photocatalyst or as a dopant, in particular in the emitting layer of light-emitting diodes (OLEDs), as well as light-emitting devices or light-emitting diodes (OLEDs) comprising such compounds.

### TECHNICAL BACKGROUND

The search for molecules having delayed fluorescence properties is at the core of developing effective luminescence devices with a low energy cost. These molecules, used pure or as dopants of emitting layers of light-emitting diodes (OLEDs or Organic Light-Emitting Diodes) make it possible to manufacture luminescence devices having a theoretical 100% internal efficiency (meaning that all of the charges injected in the form of current is returned in light form) against only 25% for a conventional fluorophore. Until then, the emitting molecules making it possible to reach such efficiencies were phosphorescent molecules involving organometal complexes using rare metals, such as iridium or platinum. For both manufacturing cost and sustainability reasons, thermally activated delayed fluorescence (TADF) molecules, which can be purely organic, therefore represent a target of choice for researchers in a field undergoing huge economic expansion, thanks to their applications for low lighting consumption and high-resolution display devices.

Moreover, the discovery of organic molecules making it possible to emit circularly polarised luminescence (CPL) is also a field in large expansion over the last few years. In this field, these are complexes bringing into play lanthanides which make it possible to obtain the best performances in terms of luminescence polarisation degree. However, a lot of research work has been currently carried out, in order to develop purely organic CPL emitting molecules to limit the manufacturing costs and to facilitate their incorporation in devices. Indeed, these small chiral molecules have a high application potential, since they can be used in the design of advanced technology devices enabling, for example, the optical storage of information, 3D display or securing a document.

Furthermore, in the field of application of OLEDs relating to the display, the molecules simultaneously having delayed fluorescence and circularly polarised luminescence properties are particularly attractive. Indeed, this type of emitters makes it possible to limit the loss of shine of screens caused by the filters used to reduce the reflection of outside light. These optical filters are most often composed of a delay waveplate (quarter waveplate) and a polariser capable of selecting the polarisation of the luminescence passing through it and to annul the reflection on the OLED of the external luminescence component. However, a significant part of the light intensity produced by the molecule in the emitting layer of the OLED is lost for a molecule not emitting circularly polarised luminescence.

The emitting molecules of circularly polarised luminescence are characterised by their quantum efficiency ϕ_{F} (the measurement of the effectiveness of the photon emission), but also by the dissymmetry factor |g_{light}| considering the amplitude of the circular polarisation. This g_{light} value is between −2 and 2, the value 0 represents an absence of circular polarisation. For a purely organic fluorophore, the |g_{light}| value is typically between 10′ and 10′. For the time being, very few organic molecules have high values of both ϕ_{F} (greater than 50% and ideally close to 100%) and |g_{light}| (ideally greater than or equal to 10⁻³). In the context of the present invention, the quantum efficiency is defined as follows:

$${ϕ}_{F} = \frac{Number of photons emitted}{Number of photons absorbed} \times 100 \qquad [ Chem 1 ]$$

Generally, the aggregation of fluorophores leads to a high decrease in the quantum efficiency via the ACQ (Aggregation-Caused Quenching) phenomenon. However, for certain fluorophores, an AIEE (Aggregation-Induced Emission Enhancement) phenomenon can be observed, where the aggregation will exacerbate the fluorescence of the compounds thus making it possible to use them at a high concentration or in pure solid form.

Moreover, the combination of TADF and AIEE properties makes it possible to consider the use of emitting layers composed only of the light-emitting molecule within an OLED device where, generally, the emitter is placed in a matrix in order to avoid fluorescence decreasing or “quenching” problems due to aggregation.

It is known that the emission by delayed fluorescence (TADF) is possible if the energy difference between the excited singlet and triplet states of lower energy (ΔE_{ST}) of the fluorescent molecule is low (less than 500 meV). The value of ΔE_{ST} is proportional to the overlap integral between the frontier orbital (FO below in the summary) HOMO and LUMO (Highest and Lowest Unoccupied Occupied Molecular Orbital) of the molecule. Thus, several molecular structures have been proposed in literature to limit the overlap between these FOs. The structure most commonly used is based on the donor-acceptor (D-A)-type molecule use, where the dihedral angle between these two entities is as close as possible to 90°. This makes it possible to overlap FOs, the HOMO being mainly located on the electron donor and LUMO on the acceptor.

G. Pieters et al. (J. Am. Chem. Soc. 2016, 138, 3990-3993) have proposed the molecule represented in [FIG. 1], wherein a chiral unit is attached to a TADF active chromophore. The chiral unit makes it possible to induce chiroptic properties (CPL). The active chromophore is constituted, in this case, of a TADF donor-acceptor system linked to a chiral unit of the BINOL type. This design has been widely exemplified by other teams with the use of other donor, acceptor or chiral patterns.

The planar chirality has also been used for the synthesis of CPTADF molecules (molecules combining CPL and TADF properties).

Today, only two examples based on derivatives of [2.2]paracyclophanes are counted, published by Zhang et al. (Org. Lett. 2018, 20, 6868) and Zysman-Colman et al. (Chem. Sci. 2019, 10, 6689), both in 2019. The first uses the paracyclophane unit both as a chirality source, and to ensure the separation between FOs: the HOMO is located on the cycle carrying amine, while the LUMO is positioned at the boron as represented in [FIG. 2]. The second uses the chirality of paracyclophane as a donor group, the HOMO-LUMO separation being done thanks to a phenyl group playing the role of a spacer disposed between the donor and the acceptor as shown in [FIG. 3]. In this example, an intrinsically chiral donor is used to generate CPL.

The need for new molecular which are simultaneously emitting via delayed fluorescence, emitting circularly polarised luminescence and having AIEE properties still subsists.

There is therefore a real need for molecules, in particular, organic molecules
thermally activated delayed fluorescence (TADF) emitters,
circularly polarised luminescence (CPL) emitters, characterised by high values, both of quantum efficiency ϕ_{F} greater than or equal to 50% in the solid state, and ideally close to 100%, and of dissymmetry factor considering the amplitude of the circular polarisation |g_{light}| (greater than or equal to 10⁻³ in solution), and
having AIEE (Aggregation-Induced Emission Enhancement) properties, where the aggregation of said molecules will exacerbate their fluorescence, this making it possible to use them at high concentration or in pure solid form, and
which are durable with a manufacturing cost that is lower than the cost for known molecules.

### SUMMARY OF THE INVENTION

The present invention relates to a compound of formula (I):

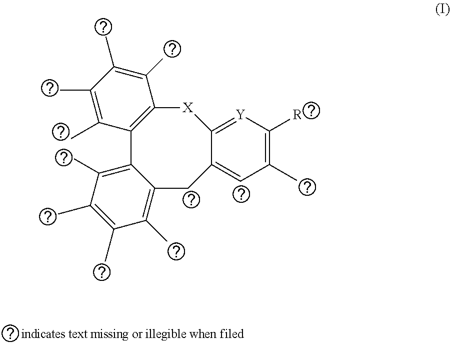

wherein:
X and X′, identical or different, represent a heteroatom chosen from the group formed by O, and N—R₉ with R₉ representing a hydrogen atom, a deuterium, an alkyl radical comprising 1 to 12 carbon atoms or an aryl radical comprising 6 to 20 carbon atoms, said alkyl and aryl radicals being optionally substituted; and
Y and Y′, identical or different, represent C—R_{y}, C—R_{y′}, a heteroatom chosen from the group formed by N, O, with R_{y} and R_{y′}, identical or different, representing a hydrogen atom, a deuterium, a nitrile group (—CN), a halogen atom chosen from the group formed by F, Cl, Br and I, an alkyl radical comprising 1 to 12 carbon atoms, an aryl radical comprising 6 to carbon atoms, a hydroxyl group, an alkoxy group, an aryloxy group, an amine group of formula NR₁₄R₁₅ with R₁₄ and R₁₅, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted; and
R_{z} and R_{z′}, identical or different, represent a hydrogen atom, a deuterium, a nitrile group (—CN), a halogen atom chosen from the group formed by F, Cl, Br and I, an alkyl radical comprising 1 to 12 carbon atoms, an aryl radical comprising 6 to 20 carbon atoms, a hydroxyl group, an alkoxy group, an aryloxy group, an amine group of formula NR₁₇R₁₈ with R₁₇ and R₁₈, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted;

or
R_{z} and R_{z′}, together with the carbon atoms to which they are bonded, form an aryl or a heterocycle chosen from the group formed by:

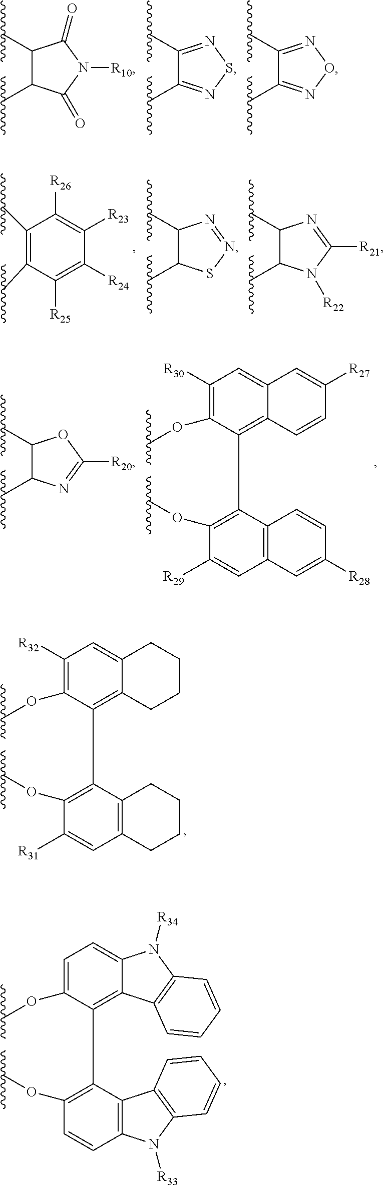

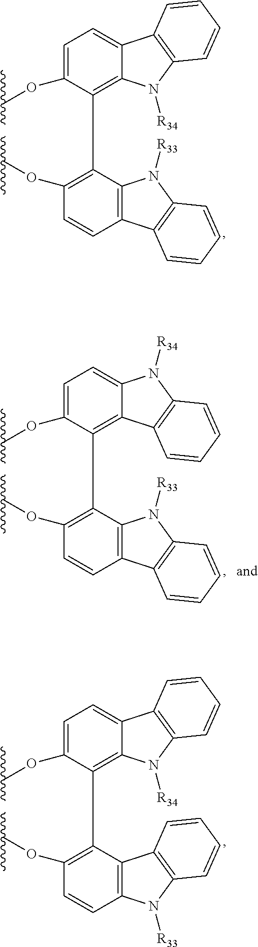

with R₁₀, R₂₀, R₂₁, R₂₂, R₂₃, R₂₄, R₂₅, R₂₆, R₂₇, R₂₈, R₂₉, R₃₀, R₃₁, R₃₂, R₃₃ and R₃₄, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms, said alkyl and aryl radicals being optionally substituted;
R₁, R₂, R₃, R₄, R₅, R₆, R₇ and R₈, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₁₂R₁₃ with R₁₂ and R₁₃, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted;

or
R₁, R₂, R₇ and R₈, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₄₀R₄₁ with R₄₀ and R₄₁, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted, and
R₃ and R₄, together with the carbon atoms to which they are bonded, form a cyclic alkyl, an aryl or a heterocycle chosen from the group formed by:

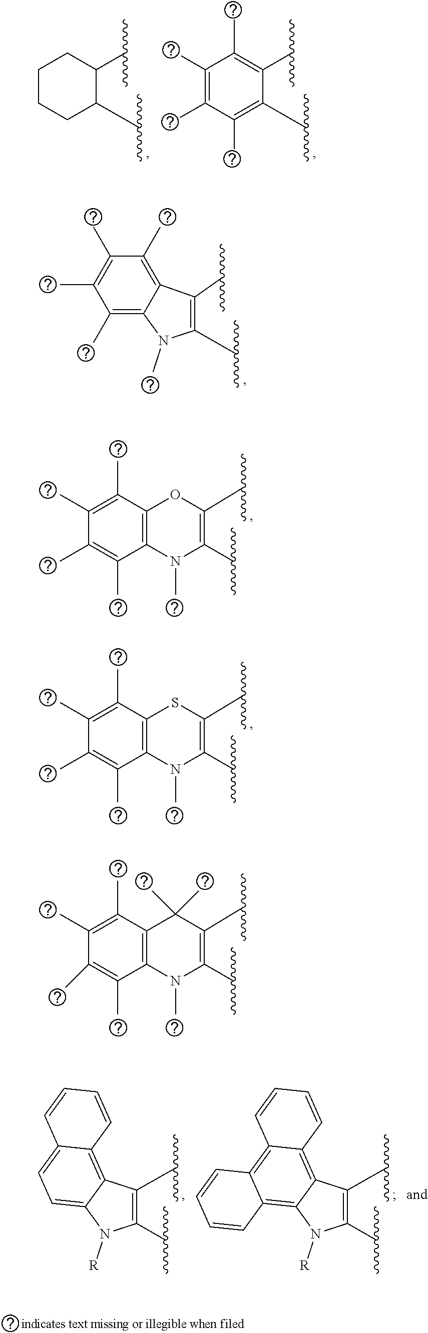

R₅ and R₆, together with the carbon atoms to which they are bonded, form a cyclic alkyl, an aryl or a heterocycle chosen from the group formed by:

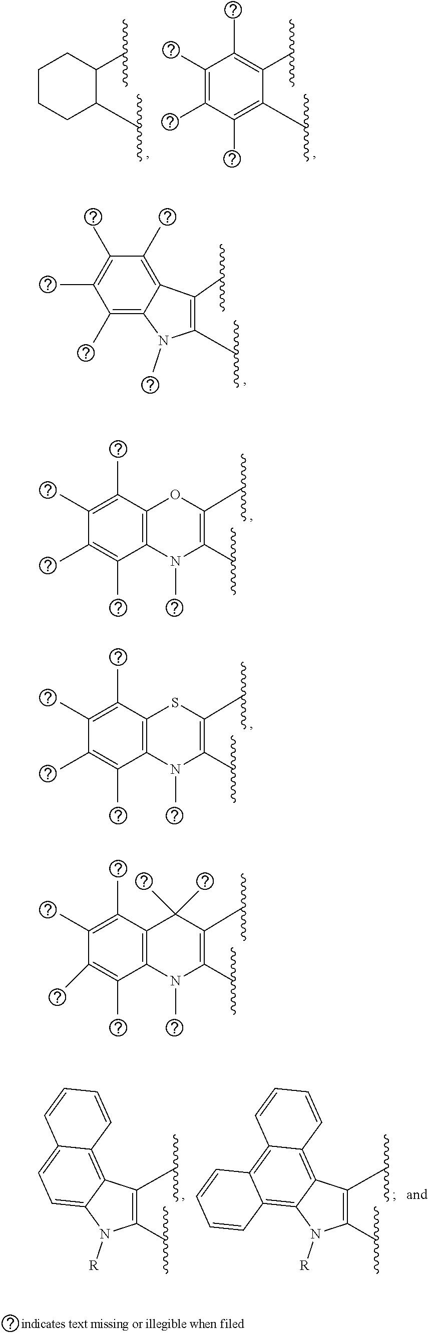

with R₁₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₃, R₆₄, R₆₅ and R₆₆, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₆₁R₆₂ with R₆₁ and R₆₂, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted;

or
R₁ and R₂, on the one hand, and R₇ and R₈, on the other hand, together with the carbon atoms to which they are bonded, each a naphthyl to lead to a fragment of the following formula:

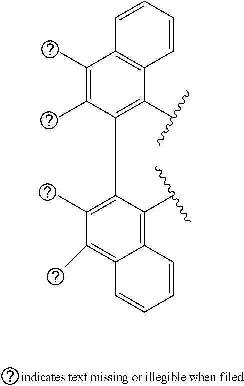 [SMILES: *c1c(-c2c(C)c(C)c3ccccc3c2*)c(C)c(C)c2ccccc12]

with
R₃, R₄, R₅ and R₆, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₁₂R₁₃ with R₁₂ and R₁₃, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted; or
R₃ and R₄, together with the carbon atoms to which they are bonded, form a cyclic alkyl, an aryl or a heterocycle chosen from the group formed by:

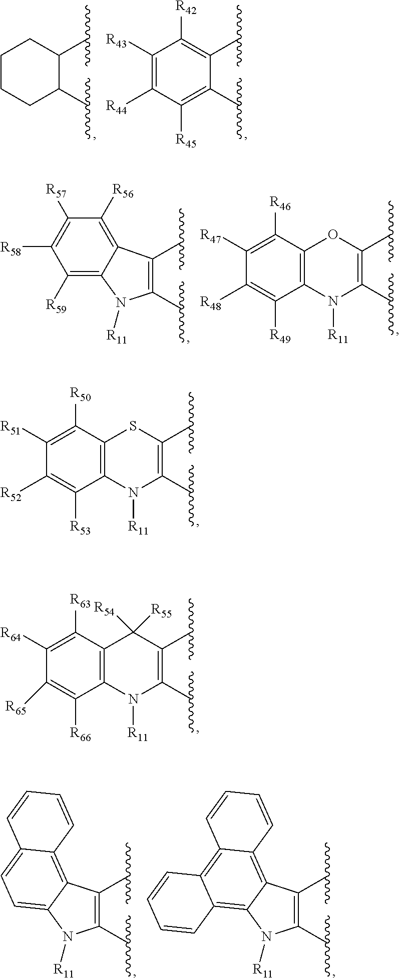

and
R₅ and R₆, together with the carbon atoms to which they are bonded, form a cyclic alkyl, an aryl or a heterocycle chosen from the group formed by:

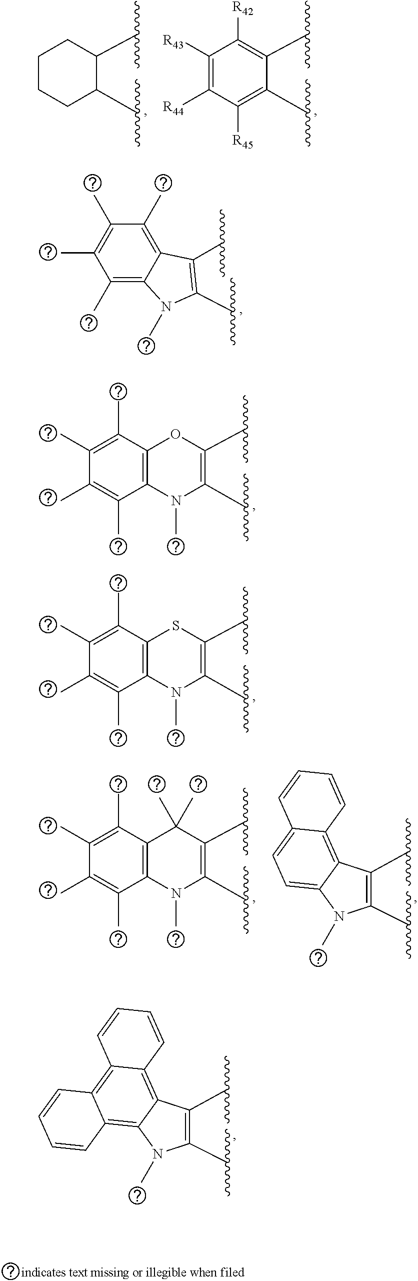

with R₁₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₃, R₆₄, R₆₅ and R₆₆, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₆₁R₆₂ with R₆₁ and R₆₂, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted;

excluding the following compounds

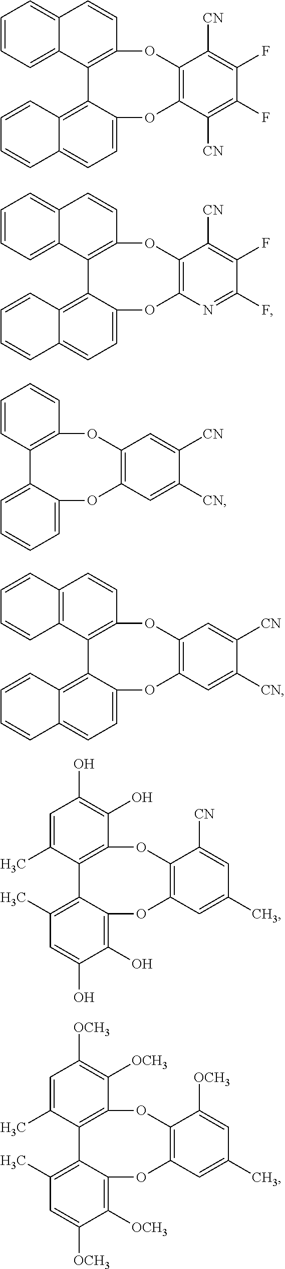

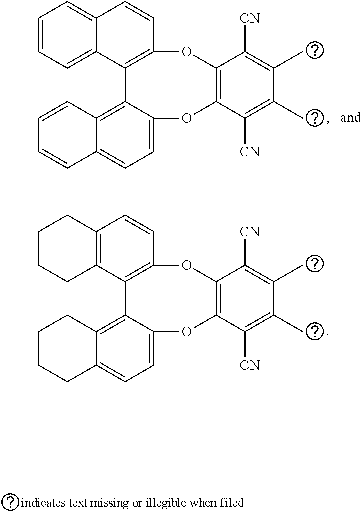 [SMILES: Cc1c(C)c(C#N)c2oc3ccc4ccccc4c3c3c(ccc4ccccc43)oc2c1C#N ; Cc1c(C)c(C#N)c2oc3ccc4c(c3c3c5c(ccc3oc2c1C#N)CCCC5)CCCC4]

The chemical structure of the compound of formula (I) extends to all possible positional and functional isomers which can be obtained by making a functional group move on the different carbons of the carbon chain, and to all possible configuration isomers which can be obtained by making the configuration of the centres, axes or individual chiral surfaces of the compound of formula (I) vary.

The invention also relates to the use of a compound of formula (I), as a photocatalyst or as a dopant, in particular in the emitting layers of light-emitting diodes (OLEDs).

It further relates to a light-emitting device or a light-emitting diode (OLED) comprising a compound of formula (I).

### BRIEF DESCRIPTION OF THE FIGURES

Other features and advantages of the invention will appear upon reading the detailed description below for the understanding of which, the accompanying drawings will be referred to, wherein:

FIG. 1 represents a molecule according to G. Pieters et al. (J. Am. Chem. Soc. 2016, 138, 3990-3993), wherein a chiral unit is attached to a TADF active chromophore. The active chromophore is constituted, in this case, of TADF donor-acceptor system linked to a BINOL-type chiral unit which makes it possible to induce chiroptic properties (CPL).

FIG. 2 represents a CPL and TADF molecule described by Zhang et al. (Org. Lett. 2018, 20, 6868) using the paracyclophane unit simultaneously as a chirality source, but also to ensure the separation between the FOs:

the HOMO is located on the cycle carrying amine, while the LUMO is positioned at the boron.

FIG. 3 represents a CPL and TADF molecule described by Zysman-Colman et al. (Chem. Sci. 2019, 10, 6689) using the chirality of paracyclophane as a donor group, the HOMO-LUMO separation being done thanks to a phenyl group playing the role of a spacer disposed between the donor and the acceptor.

FIG. 4 represents the different possible methods of deexcitation of the excited state 51 after absorption of a photon. The different phenomena present after excitation with their respective lifetime are represented.

The three radiative deexcitation processes presented,
prompt fluorescence, i.e. the radiative deexcitation coming from the relaxation from the lower-energy excited singlet state,
phosphorescence, and
delayed fluorescence, i.e. the radiative deexcitation coming from the relaxation from the lower-energy excited singlet state preceded by an intersystem crossing (and reverse intersystem) between the lower-energy singlet and triplet states, are luminescence manifestations. When the excitation is luminous, this is photoluminescence.

FIG. 5 represents a fluorescence decay (after degassing with argon) of B2-CNPyrF₂. The quantum efficiencies have been measured in toluene.

Decayed spectrum: in the abscissa axis, time in ns is represented, in the ordinate axis, the fluorescence intensity in counts per second is represented.

The quantum efficiency which measures the effectiveness of the photo emission, is calculated relatively with respect to an adapted reference, the absorbance and fluorescence spectra of which overlap with those of the compound studied. The formula used is as follows:

$${ϕ}_{PL} = \frac{S}{{S}_{ref}} \times \frac{1 - {10}^{- {A}_{ref}}}{1 - {10}^{- A}} \times {( \frac{n}{{n}_{ref}} )}^{2} \times {ϕ}_{ref}$$

where S represents the surface area under the emission curve of the compound to be studied and S_{ref}, the surface area under the emission curve of the reference, A and A_{ref}, the absorbances of the compound to be studied and of the reference respectively, n and n_{ref} are the refraction indices of the mediums wherein the molecule of interest and the reference are located. Finally, ϕ_{ref} is the quantum efficiency of the reference.

FIG. 6 represents the fluorescence decay of B1-TPNF₂ (in air (O₂) and after degassing with argon). The abscissa axis corresponds to time expressed in μs, and the ordinate axis corresponds to the fluorescence intensity expressed in counts per second. The quantum efficiencies have been measured in toluene. The quantum efficiency is calculated as indicated above.

### DETAILED DESCRIPTION OF THE INVENTION

The present invention aims to meet the needs identified above by providing a compound of formula (I):

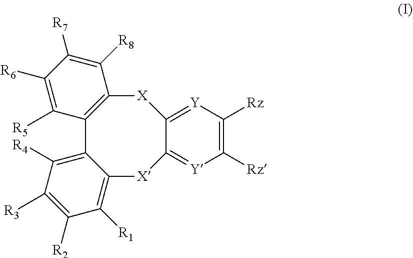

wherein
X and X′, identical or different, represent a heteroatom chosen from the group formed by O, and N—R₉ with R₉ representing a hydrogen atom, a deuterium, an alkyl radical comprising 1 to 12 carbon atoms or an aryl radical comprising 6 to 20 carbon atoms, said alkyl and aryl radicals being optionally substituted; and
Y and Y′, identical or different, represent C—R_{y}, C—R_{y′}, a heteroatom chosen from the group formed by N, O, with R_{y} and R_{y′}, identical or different, representing a hydrogen atom, a deuterium, a nitrile group (—CN), a halogen atom chosen from the group formed by F, Cl, Br and I, an alkyl radical comprising 1 to 12 carbon atoms, an aryl radical comprising 6 to carbon atoms, a hydroxyl group, an alkoxy group, an aryloxy group, an amine group of formula NR₁₄R₁₅ with R₁₄ and R₁₅, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted; and
R_{z} and R_{z′}, identical or different, represent a hydrogen atom, a deuterium, a nitrile group (—CN), a halogen atom chosen from the group formed by F, Cl, Br and I, an alkyl radical comprising 1 to 12 carbon atoms, an aryl radical comprising 6 to 20 carbon atoms, a hydroxyl group, an alkoxy group, an aryloxy group, an amine group of formula NR₁₇R₁₈ with R₁₇ and R₁₈, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted;

or
R_{z} and R_{z′}, together with the carbon atoms to which they are bonded, form an aryl or a heterocycle chosen from the group formed by:

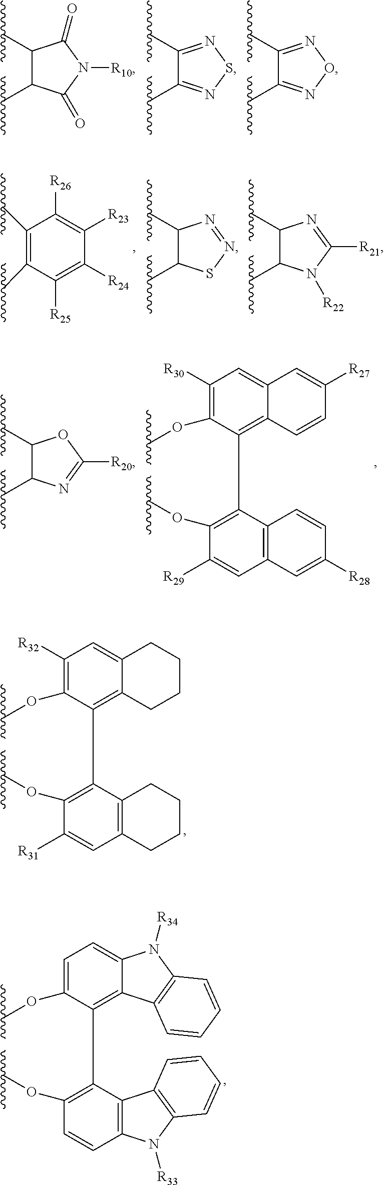

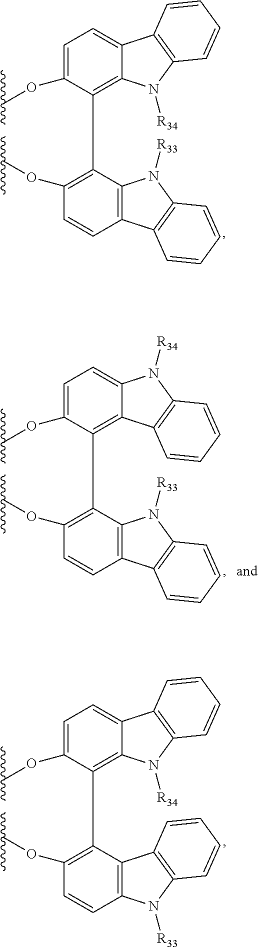

with R₁₀, R₂₀, R₂₁, R₂₂, R₂₃, R₂₄, R₂₅, R₂₆, R₂₇, R₂₈, R₂₉, R₃₀, R₃₁, R₃₂, R₃₃ and R₃₄, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted;
R₁, R₂, R₃, R₄, R₅, R₆, R₇ and R₈, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₁₂R₁₃ with R₁₂ and R₁₃, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted;

or
R₁, R₂, R₇ and R₈, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₄₀R₄₁ with R₄₀ and R₄₁, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted, and
R₃ and R₄, together with the carbon atoms to which they are bonded, form a cyclic alkyl, an aryl or a heterocycle chosen from the group formed by:

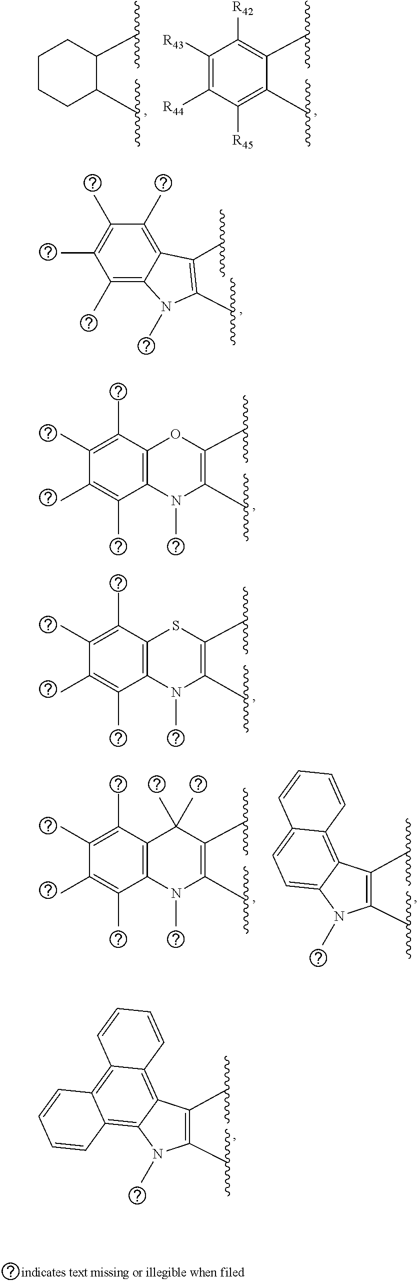

and
R₅ and R₆, together with the carbon atoms to which they are bonded, form a cyclic alkyl, an aryl or a heterocycle chosen from the group formed by:

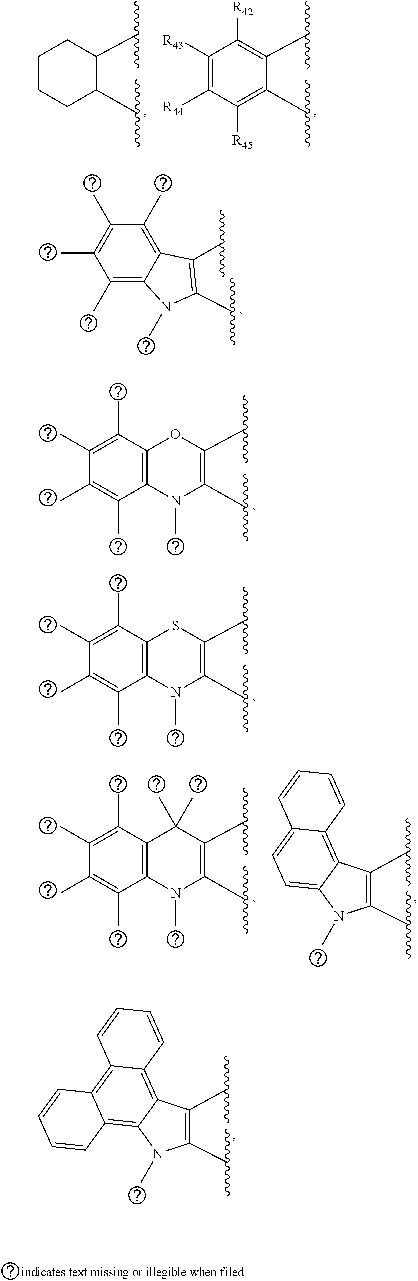

with R₁₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₃, R₆₄, R₆₅ and R₆₆, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₆₁R₆₂ with R₆₁ and R₆₂, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted; or
R₁ and R₂, on the one hand, and R₇ and R₈, on the other hand, together with the carbon atoms to which they are bonded, each a naphthyl to lead to a fragment of the following formula:

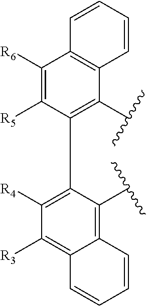

with
R₃, R₄, R₅ and R₆, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₁₂R₁₃ with R₁₂ and R₁₃, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted; or
R₃ and R₄, together with the carbon atoms to which they are bonded, form a cyclic alkyl, an aryl or a heterocycle chosen from the group formed by:

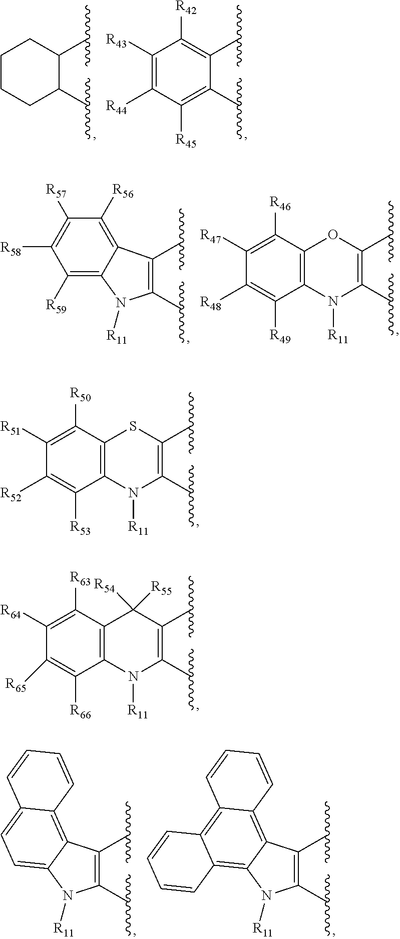

and
R₅ and R₆, together with the carbon atoms to which they are bonded, form a cyclic alkyl, an aryl or a heterocycle chosen from the group formed by:

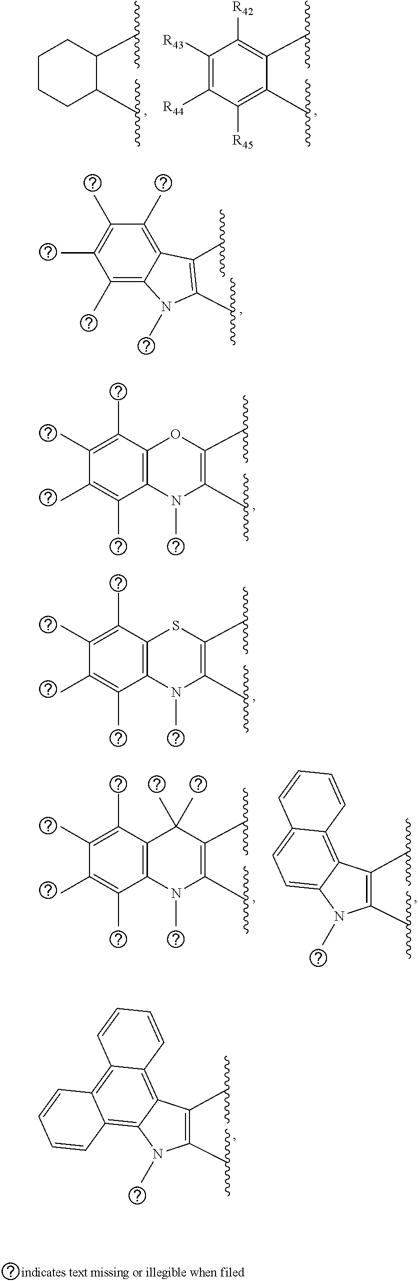

with R₁₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₃, R₆₄, R₆₅ and R₆₆, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₆₁R₆₂ with R₆₁ and R₆₂, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted;

excluding the following compounds

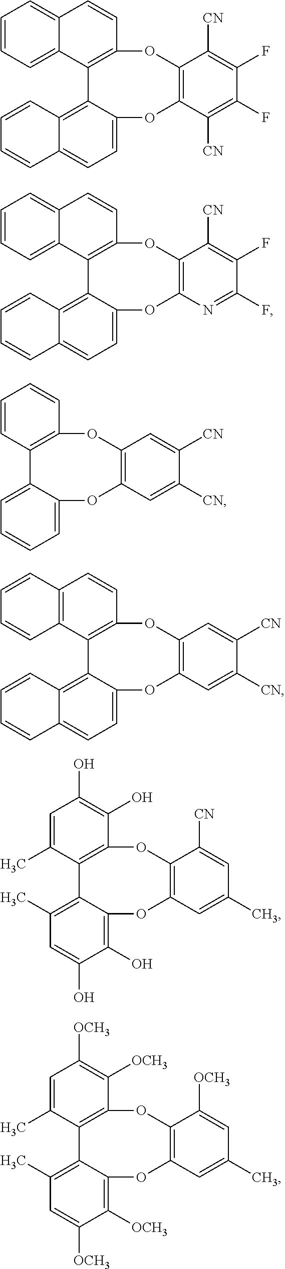

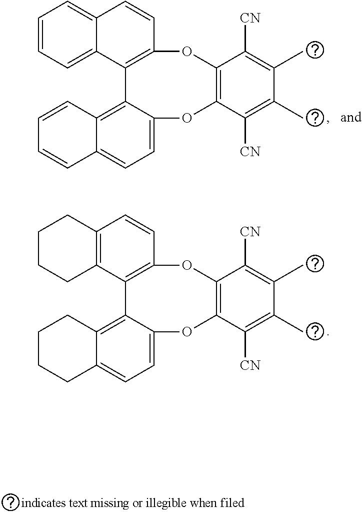 [SMILES: Cc1c(C)c(C#N)c2oc3ccc4ccccc4c3c3c(ccc4ccccc43)oc2c1C#N ; Cc1c(C)c(C#N)c2oc3ccc4c(c3c3c5c(ccc3oc2c1C#N)CCCC5)CCCC4]

The chemical structure of the compound of formula (I) extends to all possible positional and functional isomers which can be obtained by making a functional group move on the different carbons of the carbon chain, and to all possible configuration isomers which can be obtained by making the configuration of the centres, axes or individual chiral surfaces of the compound of formula (I) vary.

The present invention therefore extends to all isomers of the compounds of formula (I), in particular to all the positional, functional and configurational isomers.

The compounds of formula (I) according to the invention have the advantage of being simultaneously, emitters via delayed fluorescence (TADF), which could emit circularly polarised luminescence (CPL) and having AIEE properties.

The inventors have observed, absolutely unexpectedly, that the use of a rigid, 8-link heterocycle, around which are disposed a polycyclic electron donor pattern and a mono- or polycyclic electron acceptor, makes it possible to generate emitting molecules by delayed fluorescence. The donor patterns are thus separated from the electron acceptor patterns by the 8-link heterocycle.

The polycyclic nature of the electron donor can enable the use of chiral molecular fragments (atropisomerism) and, due to this, make it possible to combine TADF properties (induced by the limited geometry of the 8-cycle which limits the overlapping between the frontier orbitals (FOs below in the summary) and CPL (generated by the chirality of the electron donor).

A great variety of compounds bringing into play various electron donors and acceptors can thus be synthetised.

In the scope of the present invention, the term “electron donor” refers to any pattern, system, reagent, molecule, compound, group, etc. comprising a substituent or a functional group having a mesomere or inductive donor effect, which includes all the excess electron heterocycles.

The term “electron acceptor” refers to any pattern, system, reagent, molecule, compound, group, etc. comprising a substituent or a functional group having a mesomere or inductive attractor effect, which includes all the deficient electron heterocycles. By “alkyl”, this means, in the sense of the present invention, a linear, branched or cyclic, saturated, optionally substituted carbon radical, comprising 1 to 12 carbon atoms, for example 1 to 8 carbon atoms, for example 1 to 6 carbon atoms. As a saturated, linear or branched alkyl, for example methyl, ethyl, propyl, butyl, pentyl, hexyl, heptyl, octyl, nonyl, decyl, undecyl, dodecanyl radicals and their branched isomers can be mentioned.

By “cyclic alkyl”, this means, in the sense of the present invention, a cyclic, saturated, optionally substituted carbon radical, comprising 3 to 12 carbon atoms, for example 3 to carbon atoms, for example 3 to 8 carbon atoms. As cyclic alkyl, cyclopropyl, cyclobutyl, cyclopentyl, cyclohexyl, cycloheptyl, cyclooctyl, bicyclo[2,1,1] hexyl, bicyclo[2,2,1] heptyl, adamantyl radicals can be mentioned.

The term “aryl” means a mono- or poly-cyclic aromatic substituent comprising 6 to 20 carbon atoms. The aryl group can comprise, for example, 6 to 10 carbon atoms. For information, phenyl, benzyl, naphthyl, binaphthyl, phenanthrenyl, pyrenyl, anthrancenyl, o-tolyl, m-tolyl, p-tolyl, mesityl, p-nitrophenyl, o-methoxyphenyl, m-methoxyphenyl and p-methoxyphenyl, o-methoxybenzyl, p-methoxybenzyl, m-methoxybenzyl, o-methylbenzyl, p-methylbenzyl and m-methylbenzyl groups can be mentioned.

The term “heterocycle” or “heterocyclic” means a mono- or poly-cyclic substituent, comprising 5 to 10 members, saturated or unsaturated, containing 1 to 3 identical or different heteroatoms, chosen from among nitrogen, oxygen or sulphur. For information, morpholinyl, piperidinyl, piperazinyl, pyrrolyl, pyrrolidinyl, pyridinyl, imidazoloidinyl, imidazonlinyl, pyrazolyl, pyrazolidinyl, pyrazinyl, tetrahydrofuranyl, tetrahydropyranyl, thianyl, oxazolinyl, ozazolidinyl, isoxazolidnyl, thiazolidinyl, isothiazolidinyl, maleimidyl, thianthrenyl, xanthenyl, carbazolyl, furazanyl, phenothiazinyl, phenazinyl, phenoxazinyl, quinoleinyl, quinoxalinyl substituents can be mentioned. Alkyl and aryl radicals and heterocycles can be optionally substituted by one or more hydroxyl groups (—OH), one or more alkoxy groups (—O-alkyl); one or more aryloxy groups (—O-aryl); one or more halogen atoms chosen from among fluorine, chlorine, bromine and iodine atoms; one or more nitro groups (—NO₂); one or more nitrile groups (—CN); one or more carbonyl groups (—CO-alkyl); one or more alkyl radicals; one or more aryl radicals; with alkyl and aryl such as defined in the scope of the present invention.

It must be noted that in all the substituents, radicals, groups, etc., mentioned and/or defined in the context of the present invention, one or more hydrogen atoms can be, optionally replaced by one or more deuterium (²H).

The representation “ [symbol] ” such as used in this case in relation to a group, a substituent or a chemical fragment, is intended to represent the covalent bond by which said group or chemical fragment is bonded covalently to another group or chemical fragment.

According to a first embodiment, in the compound of formula (I)
X, X′, R₁, R₂, R₃, R₄, R₅, R₆, R₇ and R₈ are such as defined above;

and
Y and Y′, identical or different, represent C—R_{y}, C—R_{y′}, with R_{y} and R_{y′}, identical or different, representing a hydrogen atom, a deuterium, a nitrile group (—CN), a halogen atom chosen from the group formed by F, Cl, Br and I, an alkyl radical comprising 1 to 12 carbon atoms, an aryl radical comprising 6 to 20 carbon atoms,

said alkyl and aryl radicals being optionally substituted; and
R_{z} and R_{z′}, identical or different, represent a hydrogen atom, a deuterium, a nitrile group (—CN), a halogen atom chosen from the group formed by F, Cl, Br and I, an alkyl radical comprising 1 to 12 carbon atoms, or an aryl radical comprising 6 to 20 carbon atoms, a hydroxyl group said alkyl and aryl radicals being optionally substituted. The substituents of the alkyl and aryl radicals can be, for example, one or more hydroxyl groups (—OH), alkoxy groups (—O-alkyl); one or more aryloxy groups (—O-aryl); one or more halogen atoms chosen from among fluorine, chlorine, bromine and iodine atoms; one or more nitro groups (—NO₂); one or more nitrile groups (—CN); one or more carbonyl groups (—CO-alkyl); one or more alkyl radicals; one or more aryl radicals; with alkyl and aryl such as defined in the scope of the present invention. Preferably, the substituents of the alkyl and aryl radicals are one or more nitro groups (—NO₂); one or more nitrile groups (—CN); one or more halogen atoms chosen from among fluorine, chlorine, bromine and iodine atoms; one or more carbonyl groups (—CO-alkyl).

In a variant of this embodiment, Y and Y′ represent C—R_{y}, C—R_{y′} with R_{y} and R_{y′}, representing a nitrile group (—CN), and R_{z} and R_{z′}, identical or different, represent a halogen atom chosen from the group formed by F and Cl.

In another variant of this embodiment, Y and Y′ represent C—R_{y}, C—R_{y′} with R_{y} and R_{y′}, identical or different, representing a halogen atom chosen from the group formed by F and Cl, and R_{z} and R_{z′}, represent a nitrile group (—CN).

In another variant of this embodiment, Y and Y′ represent C—R_{y}, C—R_{y′} with R_{y} and R_{y′}, identical or different, representing a hydrogen atom, a deuterium, an aryl radical comprising 6 to 20 carbon atoms, said aryl radical being optionally substituted as indicated above, and R_{z} and R_{z′}, represent a nitrile group (—CN).

As an example of this first embodiment, the following fragments can be mentioned:

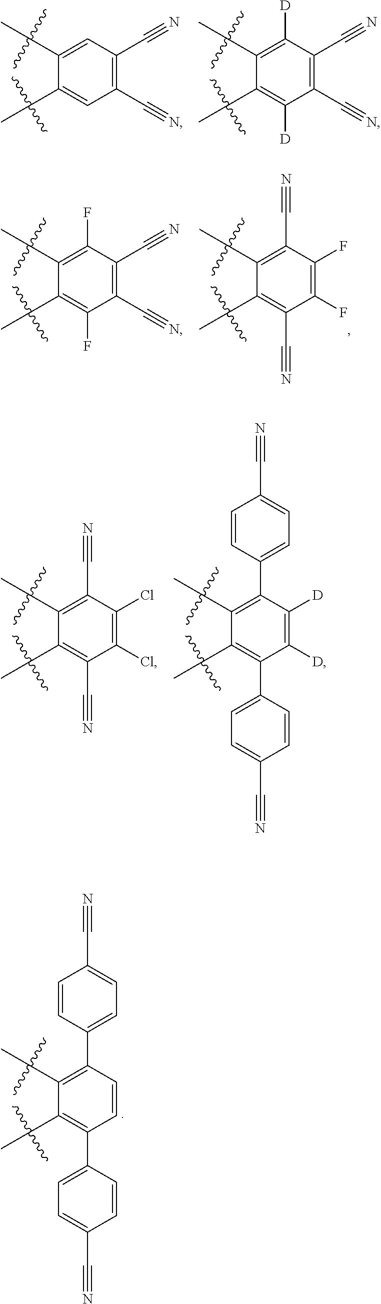

According to a second embodiment, in the compound of formula (I)
X, X′, R₁, R₂, R₃, R₄, R₅, R₆, R₇ and R₈ are such as defined above;

and
Y and Y′, identical or different, represent C—R_{y}, C—R_{y′}, a heteroatom chosen from the group formed by N, O, with R_{y} and R_{y′}, identical or different, represent a hydrogen atom, a deuterium, a nitrile group (—CN), a halogen atom chosen from the group formed by F and Cl, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted; and
R_{z} and R_{z′}, together with the carbon atoms to which they are bonded, form an aryl or a heterocycle chosen from the group formed by:

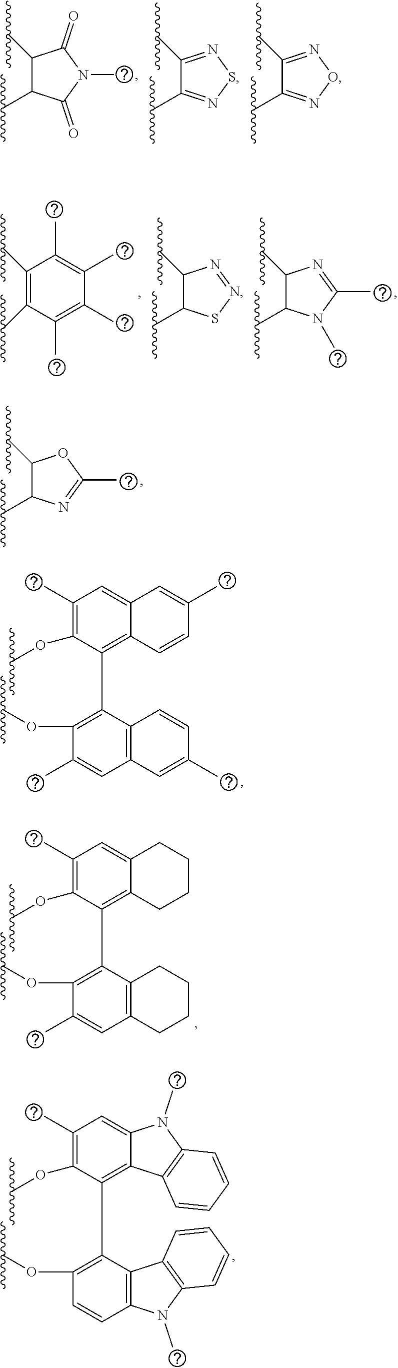

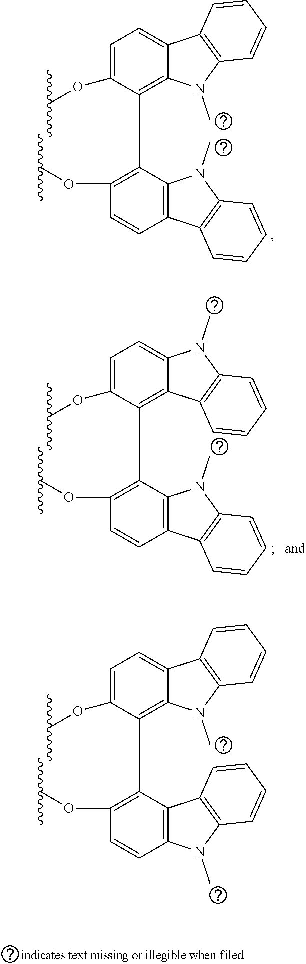 [SMILES: *Oc1ccc2c3ccccc3n(C)c2c1-c1c(O*)ccc2c3ccccc3n(C)c12 ; *Oc1ccc2c(c1-c1c(O*)ccc3c4ccccc4n(C)c13)c1ccccc1n2C ; *Oc1ccc2c(c1-c1c(O*)ccc3c4ccccc4n(C)c13)c1ccccc1n2C]

with —R₁₀, R₂₀, R₂₁, R₂₂, R₂₃, R₂₄, R₂₅, R₂₆, R₂₇, R₂₈, R₂₉, R₃₀, R₃₁, R₃₂, R₃₃ and R₃₄, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted.

In a variant of this embodiment, R₁₀, R₂₀, R₂₁, R₂₂, R₂₃, R₂₄, R₂₅, R₂₆, R₂₇, R₂₈, R₂₉, R₃₀, R₃₁, R₃₂,

R₃₃ and R₃₄, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms,

Said alkyl and aryl radicals being optionally substituted.

The alkyl radical can be, for example, a methyl, ethyl, propyl, butyl, pentyl, hexyl radical, and their branched isomers.

The aryl radical can be, for example, a phenyl, benzyl, naphthyl, phenanthrenyl radical. The substituents of the alkyl and aryl radicals can be, for example, one or more hydroxyl groups (—OH), one or more alkoxy groups (—O-alkyl); one or more aryloxy groups (—O-aryl); one or more halogen atoms chosen from among fluorine, chlorine, bromine and iodine atoms; one or more nitro groups (—NO₂); one or more nitrile groups (—CN); one or more carbonyl groups (—CO-alkyl); one or more alkyl radicals; one or more aryl radicals; with alkyl and aryl such as defined in the scope of the present invention. Preferably, the substituents of the alkyl and aryl radicals are one or more nitro groups (—NO₂); one or more nitrile groups (—CN); one or more halogen atoms chosen from among fluorine, chlorine, bromine and iodine atoms; one or more carbonyl groups (—CO-alkyl).

In a variant of this embodiment,
Y and Y′, identical or different, represent C—R_{y}, C—R_{y′}, a heteroatom chosen from the group formed by N, O, with R_{y} and R_{y′}, identical or different, being a hydrogen atom, a deuterium, a nitrile group (—CN), a halogen atom chosen from the group formed by F, Cl, an amine group of formula NR₃₅R₃₆ with R₃₅ and R₃₆, identical or different, representing a hydrogen atom, a deuterium, an alkyl radical comprising 1 to 12 carbon atoms, or an aryl radical comprising 6 to 20 carbon atoms said alkyl and aryl radicals being optionally substituted; and

R_{z} and R_{z′}, together with the carbon atoms to which they are bonded, form:

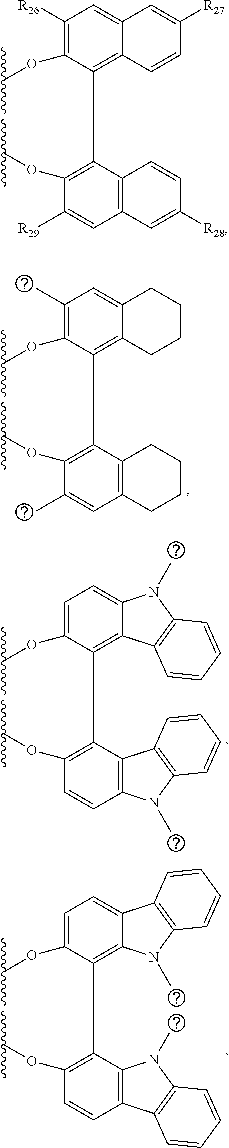

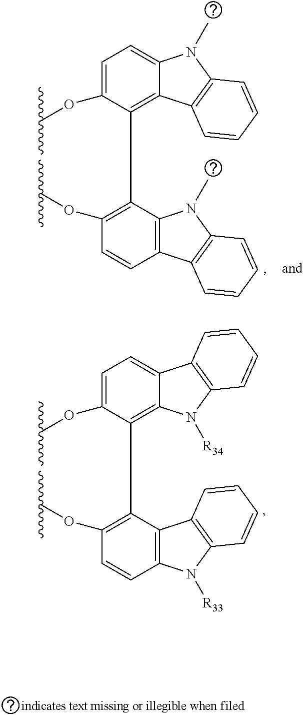

with R₂₇, R₂₈, R₂₉, R₃₀, R₃₁, R₃₂, R₃₃ and R₃₄, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted as indicated above.

In another variant of this embodiment,
Y and Y′, identical or different, represent a heteroatom chosen from the group formed by N and O, C—R_{y}, C(R_{y′}) with R_{y} and R_{y′}, identical or different, representing a hydrogen atom, a deuterium, a nitrile group (—CN), a halogen atom chosen from the group formed by F and Cl, an alkyl radical comprising 1 to 12 carbon atoms, an aryl radical comprising 6 to 20 carbon atoms, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted; and
R_{z} and R_{z′}, together with the carbon atoms to which they are bonded, form a heterocycle chosen from the group formed by:

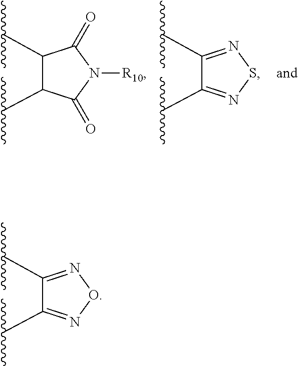

with R₁₀ representing a hydrogen atom, a deuterium, an alkyl radical comprising 1 to 12 carbon atoms, an aryl radical comprising 6 to 20 carbon atoms, said alkyl and aryl radicals being optionally substituted as indicated above. For example, R₁₀ represents a hydrogen atom, a deuterium, or a methyl, ethyl, propyl, butyl radical, and their branched isomers, or a phenyl, benzyl or naphthyl radical.

In this other variant, for example, Y and Y′, identical or different, represent C—R_{y}, C(R_{y′}) with R_{y} and R_{y′}, identical or different, being a hydrogen atom, a deuterium, a nitrile group (—CN), a halogen atom chosen from the group formed by F and Cl, an alkyl radical comprising 1 to 12 carbon atoms, an aryl radical comprising 6 to 20 carbon atoms, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted.

In a preferred embodiment of this other variant, R_{z} and R_{z′}, together with the carbon atoms to which they are bonded, form

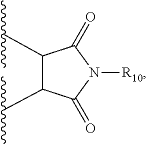

with R₁₀ such as defined above. Preferably, R₁₀ is a phenyl. As an example of this second embodiment, the following fragments can be mentioned:

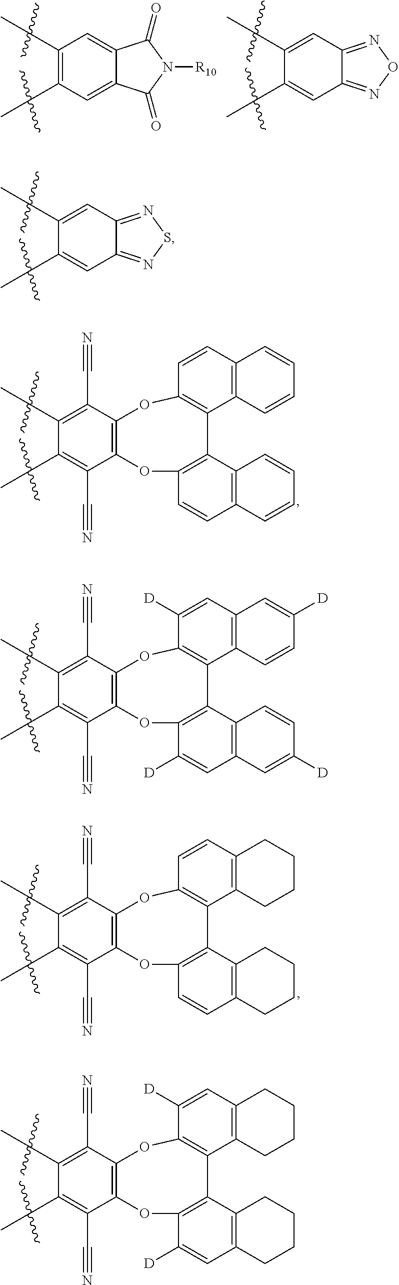

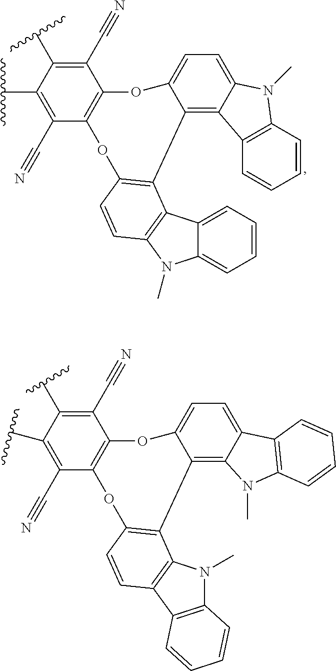 [SMILES: *c1c(*)c(C#N)c2oc3ccc4c(c5ccccc5n4C)c3c3c(ccc4c3c3ccccc3n4C)oc2c1C#N ; *c1c(*)c(C#N)c2oc3ccc4c5ccccc5n(C)c4c3c3c(ccc4c5ccccc5n(C)c43)oc2c1C#N]

with R₁₀ such as defined below in the scope of this second embodiment and of these variants.

According to a third embodiment, in the compound of formula (I)
X, X′, R₁, R₂, R₃, R₄, R₅, R₆, R₇ and R₈ are such as defined above;

and
Y and Y′, identical or different, represent a heteroatom chosen from the group formed by N, O, C—R_{y}, C—R_{y′} with R_{y} and R_{y′}, identical or different, represent a hydrogen atom, a deuterium, a nitrile group (—CN); and
R_{z} and R_{z′}, identical or different, represent a hydrogen atom, a deuterium, a nitrile group (—CN), a halogen atom chosen from the group formed by F and Cl.

In a variant of this third embodiment, Y represents C—R_{y} with R_{y} being a nitrile group (—CN), Y′ represents N, and R_{z} and R_{z′}, identical or different, represent a halogen atom chosen from the group formed by F and Cl.

As an example of this third embodiment, the following fragment can be mentioned:

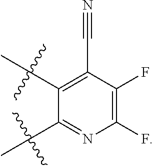 [SMILES: *c1nc(F)c(F)c(C#N)c1*]

According to a fourth embodiment, in the compound of formula (I)
X, X′, R₁, R₂, R₃, R₄, R₅, R₆, R₇ and R₈ are such as defined above;

and
Y and Y′, identical or different, represent a heteroatom chosen from the group formed by N and O; and
R_{z} and R_{z′}, together with the carbon atoms to which they are bonded, form:

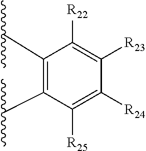

with R₂₃, R₂₄, R₂₅ and R₂₆, identical or different, represent a hydrogen atom, a deuterium, an alkyl radical comprising 1 to 12 carbon atoms, an aryl radical comprising 6 to 20 carbon atoms, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted.

The alkyl radical can be, for example, a methyl, ethyl, propyl, butyl, pentyl, hexyl radical, and their branched isomers.

The aryl radical can be, for example, a phenyl, benzyl, naphthyl, phenanthrenyl radical. The substituents of the alkyl and aryl radicals can be, for example, one or more hydroxyl groups (—OH), one or more alkoxy groups (—O-alkyl); one or more aryloxy groups (—O-aryl); one or more halogen atoms chosen from among fluorine, chlorine, bromine and iodine atoms; one or more nitro groups (—NO₂); one or more nitrile groups (—CN); one or more carbonyl groups (—CO-alkyl); one or more alkyl radicals; one or more aryl radicals; with alkyl and aryl such as defined in the scope of the present invention. Preferably, the substituents of the alkyl and aryl radicals are one or more nitro groups (—NO₂); one or more nitrile groups (—CN); one or more halogen atoms chosen from among fluorine, chlorine, bromine and iodine atoms; one or more carbonyl groups (—CO-alkyl).

In a variant of this fourth embodiment, Y and Y′ represent N and R_{z} and R_{z′}, together with the carbon atoms to which they are bonded, form:

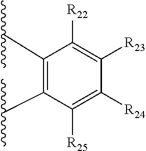

with R₂₃, R₂₄, R₂₅ and R₂₆, identical or different, represent a hydrogen atom, a deuterium, an alkyl radical comprising 1 to 12 carbon atoms, an aryl radical comprising 6 to 20 carbon atoms,

said alkyl and aryl radicals being optionally substituted.

The alkyl radical can be, for example, a methyl, ethyl, propyl, butyl, pentyl, hexyl radical, and their branched isomers.

The aryl radical can be, for example, a phenyl, benzyl, naphthyl radical.

As an example of this fourth embodiment, the following fragment can be mentioned:

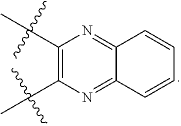 [SMILES: *c1nc2ccccc2nc1*]

According to a fifth embodiment, in the compound of formula (I)
X, X′, Y, Y′, R_{z} and R_{z′}, are such as defined above; and
R₁, R₂, R₃, R₄, R₅, R₆, R₇ and R₈, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₁₂R₁₃ with R₁₂ and R₁₃, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted.

The substituents of the alkyl and aryl radicals can be, for example, one or more hydroxyl groups (—OH), one or more alkoxy groups (—O-alkyl); one or more aryloxy groups (—O-aryl); one or more halogen atoms chosen from among fluorine, chlorine, bromine and iodine atoms; one or more nitro groups (—NO₂); one or more nitrile groups (—CN); one or more carbonyl groups (—CO-alkyl); one or more alkyl radicals; one or more aryl radicals; with alkyl and aryl such as defined in the scope of the present invention. Preferably, the substituents of alkyl and aryl radicals are one or more nitro groups (—NO₂); one or more nitrile groups (—CN); one or more halogen atoms chosen from among fluorine, chlorine, bromine and iodine atoms; one or more carbonyl groups (—CO-alkyl).

In a variant of this fifth embodiment,
X and X′ representing O; and
Y, Y′, R_{z} and R_{z′} are such as defined above; and
R₁, R₂, R₃, R₄, R₅, R₆, R₇ and R₈, identical or different, represent a hydrogen atom, a deuterium, an alkyl radical comprising 1 to 12 carbon atoms, an amine group of formula NR₁₂R₁₃ with R₁₂ and R₁₃, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted as indicated above.

In this variant, the alkyl radical is, for example, a methyl, ethyl, propyl, butyl radical, and their branched isomers.

The aryl radical can be, for example, a phenyl, benzyl, naphthyl, phenanthrenyl radical. As an example of this fifth embodiment, the following fragments can be mentioned:

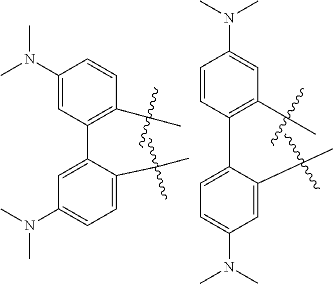

According to a sixth embodiment, in the compound of formula (I)
X and X′, identical or different, represent a heteroatom chosen from the group formed by O, and N—R₉ with R₉ representing a hydrogen atom, a deuterium, an alkyl radical comprising 1 to 12 carbon atoms or an aryl radical comprising 6 to 20 carbon atoms, said alkyl and aryl radicals being optionally substituted; and
Y, Y′, R_{z} and R_{z′} are such as defined above; and
R₁, R₂, R₇ and R₈, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₄₀R₄₁ with R₄₀ and R₄₁, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted, and
R₃ and R₄, together with the carbon atoms to which they are bonded, form a cyclic alkyl or an aryl chosen from the group formed by:

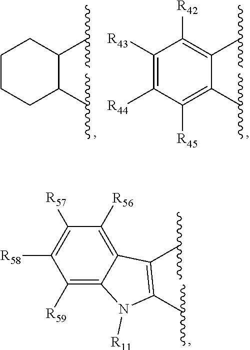

and
R₅ and R₆, together with the carbon atoms to which they are bonded, form a cyclic alkyl or an aryl chosen from the group formed by:

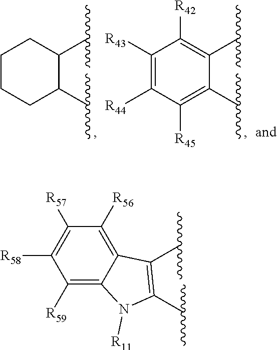

with
R₁₁, R₄₂, R₄₃, R₄₄, R₄₅, R₅₆, R₅₇, R₅₈ and R₅₉, identical or different, representing a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₆₁R₆₂ with R₆₁ and R₆₂, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted.

The substituents of the alkyl and aryl radicals can be, for example, one or more hydroxyl groups (—OH), one or more alkoxy groups (—O-alkyl); one or more aryloxy groups (—O-aryl); one or more halogen atoms chosen from among fluorine, chlorine, bromine and iodine atoms; one or more nitro groups (—NO₂); one or more nitrile groups (—CN), one or more carbonyl groups (—CO-alkyl); one of more alkyl radicals, one or more aryl radicals, with alkyl, and aryl such as defined in the scope of the present invention. Preferably, the substituents of the alkyl and aryl radicals are one or more nitro groups (—NO₂); one or more nitrile groups (—CN); one or more halogen atoms chosen from among fluorine, chlorine, bromine and iodine atoms; one or more carbonyl groups (—CO-alkyl).

In a variant of this sixth embodiment,
X and X′, identical or different, represent a heteroatom chosen from the group formed by O, and N—R₉ with R₉ representing a hydrogen atom, a deuterium, alkyl radical comprising 1 to 12 carbon atoms or an aryl radical comprising 6 to 20 carbon atoms, said alkyl and aryl radicals being optionally substituted as indicated above;
Y, Y′, R_{z} and R_{z′} are such as defined above;
R₁, R₂, R₇ and R₈, identical or different, represent a hydrogen atom, a deuterium, an alkyl radical comprising 1 to 12 carbon atoms, or an aryl radical comprising 6 to 20 carbon atoms, said alkyl and aryl radicals being optionally substituted, and
R₃ and R₄, together with the carbon atoms to which they are bonded, form a cyclic alkyl or an aryl chosen from the group formed by:

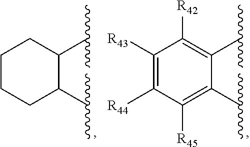

and
R₅ and R₆, together with the carbon atoms to which they are bonded, form a cyclic alkyl or an aryl chosen from the group formed by:

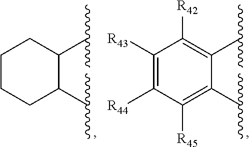

with R₄₂, R₄₃, R₄₄ and R₄₅, identical or different, representing a hydrogen atom, a deuterium, an alkyl radical comprising 1 to 12 carbon atoms, an aryl radical comprising 6 to 20 carbon atoms,

said alkyl and aryl radicals being optionally substituted as indicated above.

In another variant of this sixth embodiment,
X and X′, identical or different, representing a heteroatom chosen from the group formed by O and N—R₉ with R₉ representing a hydrogen atom, a deuterium, an alkyl radical comprising 1 to 12 carbon atoms or an aryl radical comprising 6 to 20 carbon atoms, said alkyl and aryl radicals being optionally substituted; and
Y, Y′, R_{z} and R_{z′}, are such as defined above; and
R₁, R₂, R₇ and R₈, identical or different, represent a hydrogen atom, a deuterium, an alkyl radical comprising 1 to 12 carbon atoms, an aryl radical comprising 6 to 20 carbon atoms, said alkyl and aryl radicals being optionally substituted as indicated above, and
R₃ and R₄, together with the carbon atoms to which they are bonded, form:

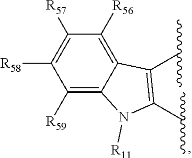

and
R₅ and R₆, together with the carbon atoms to which they are bonded, form:

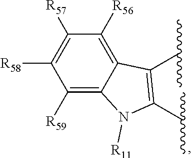

with
R₁₁, R₅₆, R₅₇, R₅₈ and R₅₃, identical or different, representing a hydrogen atom, a deuterium, an alkyl radical comprising 1 to 12 carbon atoms, an aryl radical comprising 6 to 20 carbon atoms, said alkyl and aryl radicals being optionally substituted.

In the variants of this embodiment, the alkyl radical can be, for example, a methyl, ethyl, propyl, butyl radical, and their branched isomers.

The aryl radical can be, for example, a phenyl, benzyl, naphthyl, phenanthrenyl radical. As an example of this sixth embodiment, the following fragments can be mentioned:

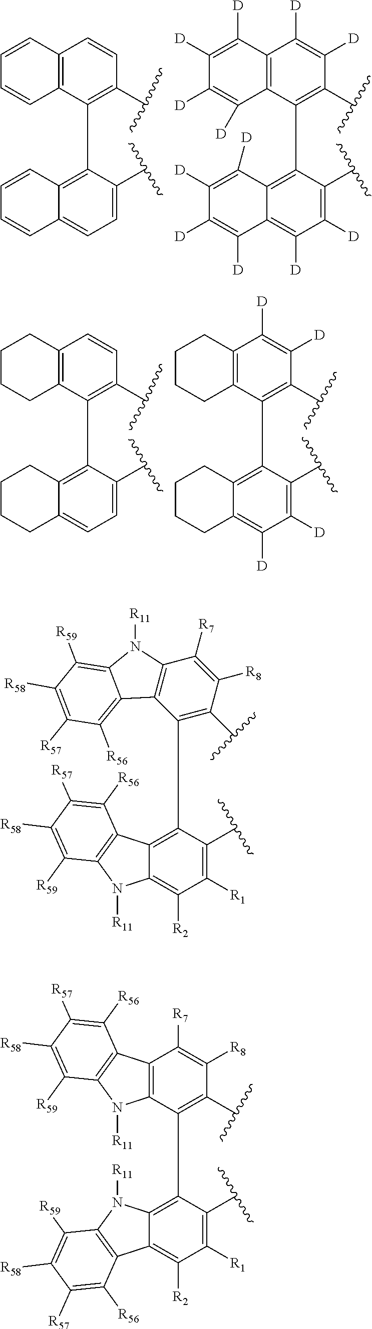

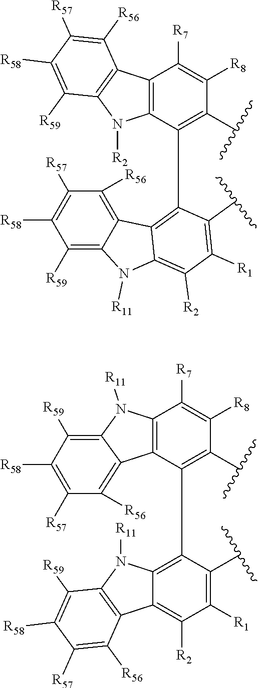

with R₁, R₂, R₇, R₈, R₁₁, R₅₆, R₅₇, R₅₈ and R₅₉ such as defined above. Preferably, R₁, R₂, R₇, R₈, R₁₁, R₅₆, R₅₇, R₅₈ and R₅₉, identical or different, represent a hydrogen atom, a deuterium, an alkyl radical chosen, for example, from the group formed by a methyl, ethyl, propyl, butyl radical, and their branched isomers, an aryl radical chosen, for example, from the group formed by a phenyl, benzyl, naphthyl and phenanthrenyl radical.

According to a seventh embodiment, in the compound of formula (I)
X and X′, identical or different, represent a heteroatom chosen from the group formed by O, and N—R₉ with R₉ representing a hydrogen atom, a deuterium, an alkyl radical comprising 1 to 12 carbon atoms or an aryl radical comprising 6 to 20 carbon atoms, said alkyl and aryl radicals being optionally substituted; and
Y, Y′, R_{z} and R_{z′} are such as defined above; and
R₁, R₂, R₇ and R₈, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₄₀R₄₁ with R₄₀ and R₄₁, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted, and
R₃ and R₄, together with the carbon atoms to which they are bonded, form an aryl or a heterocycle chosen from the group formed by:

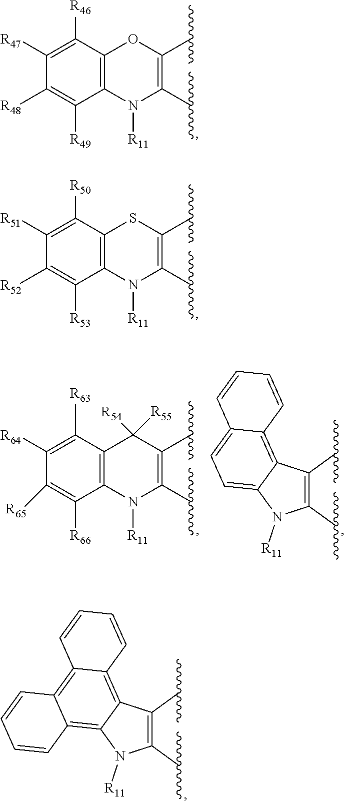

R₅ and R₆, together with the carbon atoms to which they are bonded, form a cyclic alkyl, an aryl or a heterocycle chosen from the group formed by:

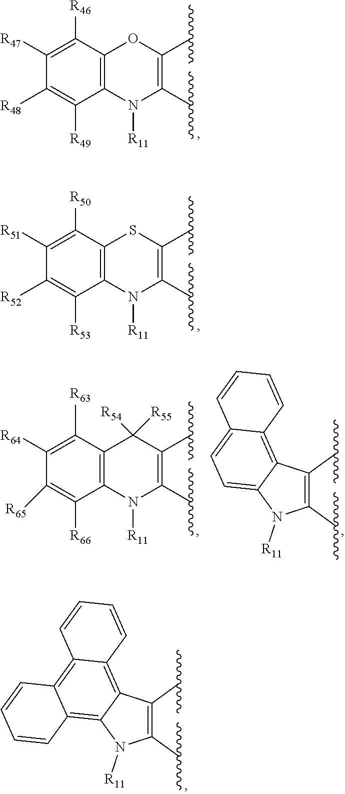

with
R₁₁, R₄₆, R₄₇, R₄₈, R₄₉, R₅₀, R₅₁, R₅₂, R₅₃, R₅₄, R₅₅, R₆₃, R₆₄, R₆₅ and R₆₆, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₆₁R₆₂ with R₆₁ and R₆₂, identical or different, representing a hydrogen atom, a deuterium, an alkyl radical comprising 1 to 12 carbon atoms or an aryl radical comprising 6 to 20 carbon atoms,

said alkyl and aryl radicals being optionally substituted.

The substituents of the alkyl and aryl radicals can be, for example, one or more hydroxyl groups (—OH), one or more alkoxy groups (—O-alkyl); one or more aryloxy groups (—O-aryl); one or more halogen atoms chosen from among fluorine, chlorine, bromine and iodine atoms; one or more nitro groups (—NO₂); one or more nitrile groups (—CN); one or more carbonyl groups (—CO-alkyl); one or more alkyl radicals; one or more aryl radicals; with alkyl and aryl such as defined in the scope of the present invention. Preferably, the substituents of the alkyl and aryl radicals are one or more nitro groups (—NO₂); one or more nitrile groups (—CN); one or more halogen atoms chosen from among fluorine, chlorine, bromine and iodine atoms; one or more carbonyl groups (—CO-alkyl).

In this variant, the alkyl radical can be, for example, a methyl, ethyl, propyl, butyl radical, and their branched isomers.

The aryl radical can be, for example, a phenyl, benzyl, naphthyl, phenanthrenyl radical. In a variant of this seventh embodiment,
X and X′, identical or different, represent a heteroatom chosen from the group formed by O, and N—R₉ with R₉ representing a hydrogen atom, a deuterium, an alkyl radical comprising 1 to 12 carbon atoms or an aryl radical comprising 6 to 20 carbon atoms, said alkyl and aryl radicals being optionally substituted as indicated above; and
Y, Y′, R_{z} and R_{z′} are such as defined above; and
R₁, R₂, R₇ and R₈, identical or different, represent a hydrogen atom, a deuterium, an alkyl radical comprising 1 to 12 carbon atoms, an aryl radical comprising 6 to 20 carbon atoms, said alkyl and aryl radicals being optionally substituted as indicated above; and
R₃, R₄, R₅ and R₆ are such as defined above; and
R₁₁, R₄₆, R₄₇, R₄₈, R₄₉, R₅₀, R₅₁, R₅₂, R₅₃, R₅₄, R₅₅, R₆₃, R₆₄, R₆₅ and R₆₆, identical or different, represent a hydrogen atom, a deuterium, an alkyl radical comprising 1 to 12 carbon atoms, an aryl radical comprising 6 to 20 carbon atoms, said alkyl and aryl radicals being optionally substituted as indicated above.

As an example of this seventh embodiment, the following fragments can be mentioned:

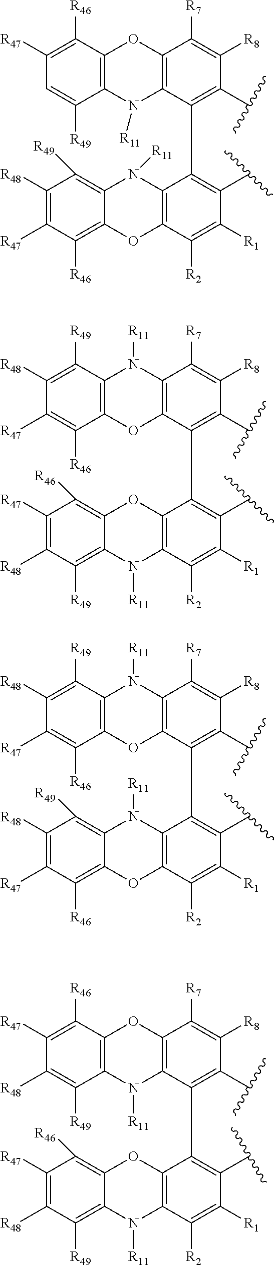

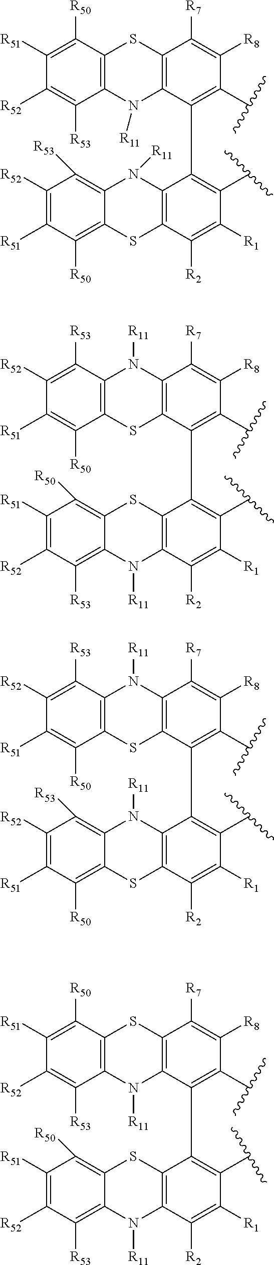

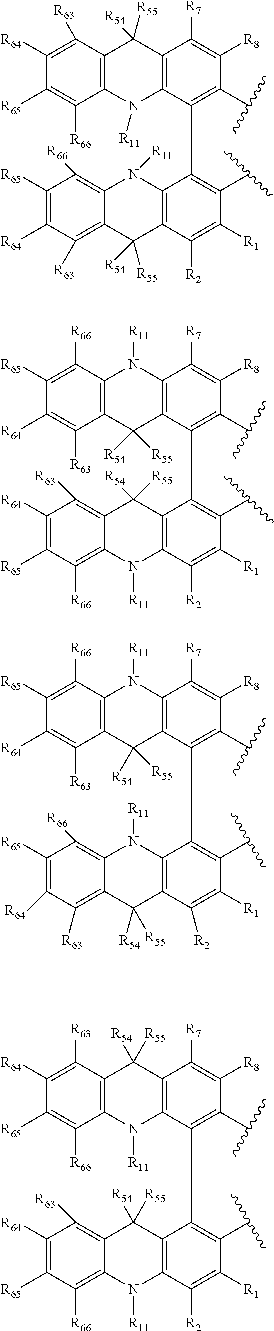

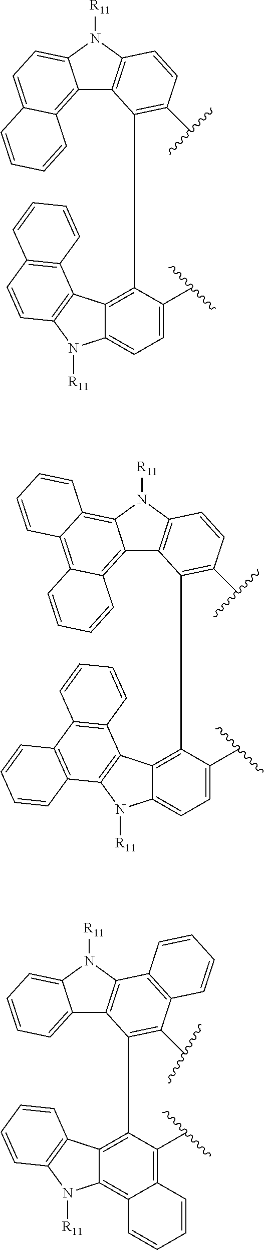

with R₁, R₂, R₇, R₈, R₁₁, R₄₆, R₄₇, R₄₈, R₄₉, R₅₀, R₅₁, R₅₂, R₅₃, R₅₄, R₅₅, R₆₃, R₆₄, R₆₅ and R₆₆, such as defined above.

Preferably, R₁, R₂, R₇, R₈, R₁₁, R₄₆, R₄₇, R₄₈, R₄₉, R₅₀, R₅₁, R₅₂, R₅₃, R₅₄, R₅₅, R₆₃, R₆₄, R₆₅ and

R₆₆, identical or different, represent a hydrogen atom, a deuterium, an alkyl radical chosen, for example, from the group formed by a methyl, ethyl, propyl, butyl radical, and their branched isomers, an aryl radical chosen, for example, from the group formed by a phenyl, benzyl, naphthyl and phenanthrenyl radical.

According to an eighth embodiment, the compound of formula (I) is chosen from the group formed by:

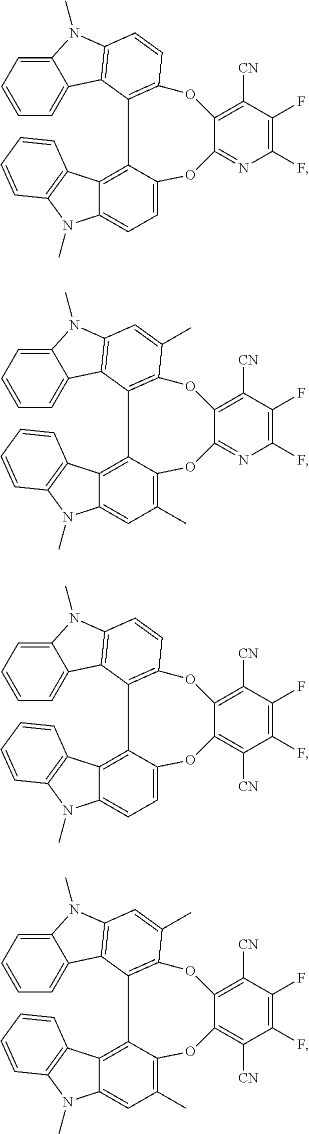

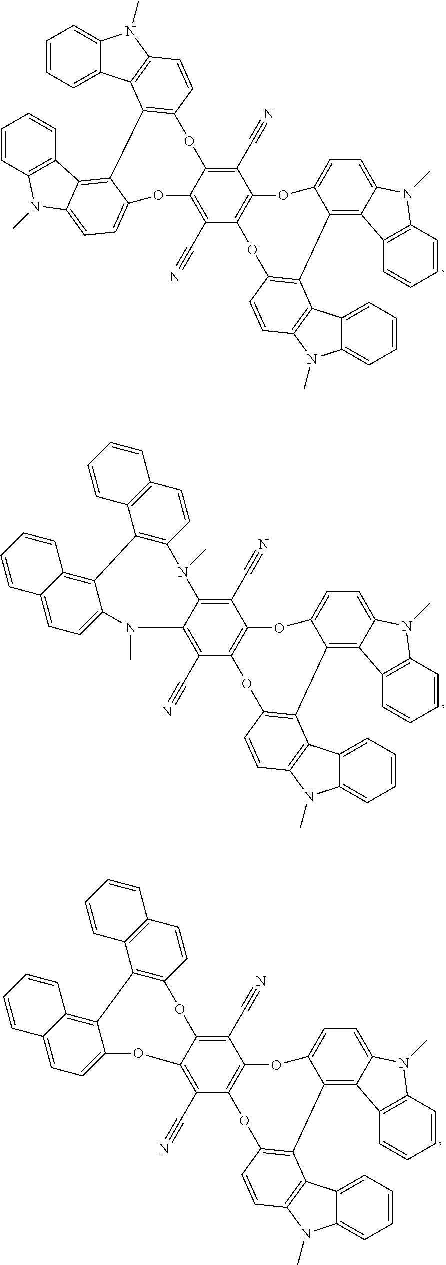 [SMILES: Cn1c2ccccc2c2c3c(ccc21)oc1c(C#N)c2oc4ccc5c(c6ccccc6n5C)c4c4c(ccc5c4c4ccccc4n5C)oc2c(C#N)c1oc1ccc2c(c4ccccc4n2C)c13 ; Cn1c2ccccc2c2c3c(ccc21)oc1c(C#N)c2c(c(C#N)c1oc1ccc4c(c5ccccc5n4C)c13)n(C)c1ccc3ccccc3c1c1c3ccccc3ccc1n2C ; Cn1c2ccccc2c2c3c(ccc21)oc1c(C#N)c2oc4ccc5ccccc5c4c4c(ccc5ccccc54)oc2c(C#N)c1oc1ccc2c(c4ccccc4n2C)c13]

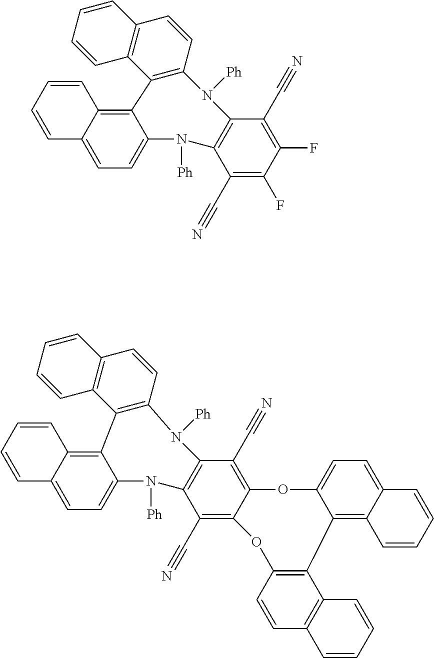 [SMILES: N#Cc1c(F)c(F)c(C#N)c2c1n(-c1ccccc1)c1ccc3ccccc3c1c1c3ccccc3ccc1n2-c1ccccc1 ; N#Cc1c2oc3ccc4ccccc4c3c3c(ccc4ccccc43)oc2c(C#N)c2c1n(-c1ccccc1)c1ccc3ccccc3c1c1c3ccccc3ccc1n2-c1ccccc1]

The compounds for formula (I) of the invention can be prepared by a method comprising the following steps:

1. The donor reagents (1 to 10 mmol) and acceptors (1 to 10 mM) are placed in a flask containing a base chosen from among Na₂CO₃, K₂CO₃ or Cs₂CO₃, NaH, and a suitable organic solvent is added, like for example, a solvent chosen from among N,N-dimethylformamide (DMF), N-methyl-2-pyrrolidone (NMP), or dimethylacetamide (DMA) in a nitrogen or argon atmosphere.

2. The reactional mixture is stirred at ambient temperature (20±5° C.) for 8 to 12 hours.

3. The reaction is stopped by adding distilled water and an organic solvent chosen, for example, from among dichloromethane (CH₂Cl₂), ethyl acetate (EtOAc), or monochloromethane (CH₃Cl). The mixture is thus extracted with an organic solvent, like for example, a solvent chosen from among dichloromethane (CH₂Cl₂), ethyl acetate (EtOAc), or monochloromethane (CH₃Cl). The combined organic phases are dried, for example, on MgSO₄, filtered and concentrated under reduced pressure to give the compound of formula (I).

The purification can be performed by any purification technique known to a person skilled in the art. Flash chromatography on silica gel by using, for example, a mixture of solvents, the solvents being chosen, for example, from among cyclohexane, naphtha, hexane, ethyl acetate, DCM, CH₃Cl, toluene) as eluent. An example of eluent is a cyclohexane/dichloromethane (1/1) mixture.

The abovementioned solvents have been, as an example, and are not in any case, limiting.

The compounds of formula (I) according to the invention, therefore make it possible to simultaneously combine the circularly polarised luminescence (CPL) emission properties and those of delayed fluorescence (TADF). These compounds also have aggregation-induced emission enhancement (AIEE) properties. The combination of these properties within one same compound has a high potential for application as a dopant in the emitting layers of organic light-emitting diodes (OLEDs). Furthermore, these compounds could also have applications in the field of photocatalysis.

The invention therefore aims for the use of a compound of formula (I) according to the invention as a photocatalyst, in particular in activation reactions of C—H bonds, formation reactions of C—C and C—X bonds with X being a heteroatom, or as a dopant, in particular in the emitting layers of organic light-emitting diodes (OLEDs).

The invention further aims for a light-emitting device or a light-emitting diode comprising a compound of formula (I) according to the invention.

### Examples

The different reagents and solvents used in the method of the invention and in the examples are, generally, commercial compounds or can be prepared by any method known to a person skilled in the art. The synthetised compounds have been characterised by RMN ¹H, RMN ¹³C, RMN ¹⁹F analysis techniques on a Bruker Avance 400 MHz spectrometer. The chemical movements are reported in parts per million (ppm) configured on the residual peaks of the deuterated solvent used and the coupling constants are indicated in Hertz (Hz). The multiplicity of peaks is referenced by singlet (s), doublet (d), triplet (t). The multiplicities which could not be interpreted are reported as multiplets (m).

The synthetised compounds have been characterised by the analysis and mass spectrometry techniques using the instrument ESI-Quadripole autopurify, Waters (pump: 2545, mass: ZQ2000) and mass spectrometry.

The visible-UV spectra have been recorded on a Cary 50 or Cary 400 (Agilent) double-beam spectrometer by using a 10 mm trajectory quartz cell.

The circular dichroism (CD) spectra have been recorded on a Jasco spectropolarimeter (model J-815) equipped with a Peltier thermostatically controlled cell carrier and an Xe laser. The data have been recorded at 20° C. by using a 1 mm×1 cm cell. The signals obtained have been processed by subtracting the contribution from the solvent and the cells.

The emission spectra have been measured on a Fluoromax-3 (Horiba) or Fluoromax-4 (Horiba) or Fluorolog (Horiba) spectrofluorimeter. A right-angle configuration has been used. The optical density of the samples has been verified as being less than 0.1 to avoid the reabsorption artefacts.

The fluorescence decay curves in the regime ns have been obtained by the time-correlated single photon counting method (TCSPC) with a femtosecond laser excitation composed of a Titanium Sapphire laser (Tsunami, Spectra-Physics) pumped by a double Nd: YVO4 laser (Millennia Xs, Spectra-Physics). The 990 nm luminous pulses of the oscillator have been selected by an acousto-optic crystal at a repetition rate of 4 MHz, then tripled to 330 nm by non-linear crystals. Fluorescence photons have been detected at 90° through a monochromator and a polariser at magic angle by means of a Hamamatsu MCP R3809U photomultiplier, connected to a TCSPC SPC-630 module from Becker & Hickl. The instrumental response function has been recorded before each decay measurement with an fwhm (full width at half maximum) of ˜25 ps. The fluorescence data have been analysed using the Globals software package developed at the Laboratory for Fluorescence Dynamics of the University of Illinois in Urbana-Champaign, which comprises a re-convolution analysis and an overall minimisation method of the non-linear least squares. The decay curves of the fluorescence μs have been obtained by using a flash laser photolysis spectrometer LP920 from Edimbourg combined with an Nd: YAG laser (Continuum) tripled to 355 nm via non-linear crystals. This third harmonic is optimised to pump an OPO which can generate a 425 nm signal. The fluorescence photons have been detected at 90° through a high-pass filter and a monochromator by means of a Hamamatsu R928 photomultiplier. The Levenberg-Marquardt algorithm has been used for the non-linear adjustment of the least squares (adjustment of the tail) such as implemented in the software L900 (Edimbourg instrument). In order to estimate the quality of the adjustment, the weighted residues have been calculated.

Circularly Polarised Luminescence (CPL) Measurements (General Points)

A molecule can preferably absorb the circularly polarised luminescence, in the same manner, it can also emit excess circularly polarised luminescence by radiative deexcitation in the form of luminescence (fluorescence, delayed fluorescence or phosphorescence). To observe this circularly polarised luminescence (CPL) phenomenon, it is necessary that fluorophore is subjected to a force field: it can come from the chiral fluorochrome studied (chiral force field intrinsic to the molecule), CPL is thus referred to, or thus if can come from an external magnetic field which is located in the propagation direction of the light emitted, in this case, the molecule studied is not necessarily chiral and MCPL is referred to (Magnetic Circularly Polarised Luminescence).

In the scope of the present invention, only the CPL will be studied; no study will be based on the MCPL properties.

Like for circular dichroism, to measure the CPL, the difference in intensity (ΔI(λ)) is measured between the emission of left circularly polarised luminescence (I_{L}(λ)) with respect to the emission of right circularly polarised luminescence (I_{R}(λ)):

ΔI(λ)=I_{L}(λ)−I_{R}(λ)

The ΔI(λ) measurements are quite complex since they can be subjected to numerous experimental artefacts (linearly polarised luminescence, birefringence phenomena) and to a problem linked to the detection limit. Indeed, generally, the proportion of circularly polarised luminescence is very low with respect to the total luminescence emitted I(λ), due to this, the photomultipliers used must be very efficient. In order to be able to compare the emission of circularly polarised luminescence between fluorophores, the luminescence dissymmetry factor must be used:

$${g}_{light} ( λ ) = 2 \frac{Δ I ( λ )}{I ( λ )}$$

where I(λ) represents the total luminescence intensity. Due to the “factor 2”, g_{light} can take values of between −2 and 2, like for g_{obs}, representing a total emission of right or left circularly polarised luminescence. In the same manner as for the CD, if the g_{light} value is zero, then the molecule does not emit excess circularly polarised luminescence.

The CPL measurements give information on the transition chiral environments associated with radiative deexcitations of the compounds and therefore lower-energy singlet or triplet states which are responsible for the fluorescence (delayed or not) and for the phosphorescence. However, during the absorption and internal conversion phenomenon (and intersystem crossing for the phosphorescence), it comes about that the molecule changes geometry with respect to its fundamental state. That is why it is possible to have a different g_{obs} of 0 and a zero g_{light} (and theoretically the opposite is also possible, but has never been observe). A loss of chiral information can occur, if the geometry of the molecule is modified, and if the radiative deexcitation transition does not bring into play the intrinsically chiral parts of the molecule.

It is also possible to theoretically determine, i.e. by modelling and calculations, the g_{light} values via the following formula:

$${g}_{light} ( λ ) = 4 R e [ \frac{μ \cdot m}{{\vert μ \vert}^{2} + {\vert m \vert}^{2}} ] = 4 cos θ \frac{\vert μ \vert \vert m \vert}{{\vert μ \vert}^{2} + {\vert m \vert}^{2}} \approx 4 cos θ \frac{\vert m \vert}{\vert μ \vert}$$

In this formula, μ and m represent respectively the electric and magnetic dipolar transition moments in the excited state, θ is the angle between these two vectors. With this formula, it is easy to understand that the greater the values of the norms of the vectors are, the greater the g_{light} value will be

$$( if θ \neq \frac{π}{2} ) .$$

Notwithstanding, in practice, m is often very small compared with μ (hence the approximation

$${g}_{light} ( λ ) \approx 4 cos θ \frac{\vert m \vert}{\vert μ \vert} ) ,$$

thus g_{light} is directly proportional to |m| and inversely proportional to |μ|. Due to this, high g_{light} values are expected for magnetically authorised and electrically prohibited transitions. That is why the CPL measurements have been mainly applied to the lanthanide complexes initially. These compounds have the particularity of being able to perform particular transitions (Laporte transition f−f, theoretically prohibited, but observable thanks to the spin-orbit coupling) which give a very high magnetic transition moment and a low electric transition moment. That is why, despite the high g_{light} values obtained (the maximum value measured up to now is 1.38), the quantum efficiencies of this type of complexes are low (around a few percent in the best case).

Delayed Fluorescence

Reverse Intersystem Crossing (rISC)

As stated above, from the state T₁, it is possible for a molecule to return to an excited singlet state S₁. This can be done by triplet-triplet annihilation, or by an intersystem crossing. In the second case, if the energy difference between the low-energy singlet state S₁ and the lower-energy triplet state T₁ (ΔE_{ST}) is quite low, reverse intersystem crossing (rISC) is referred to, the following transition is thus achieved:

T₁→S₁.

Like the intersystem crossing, the reverse intersystem crossing can only occur if the initial state (T₁) has a sufficiently long lifespan, and that the speed of the reverse intersystem crossing is sufficiently high.

This phenomenon is energetically dependent, since it is moved from the low-energy state (T₁) to the high-energy state (S₁). Thus, it is considered that such that there is spontaneously, at ambient temperature (20 s 5° C.), of the reverse intersystem crossing, the energy difference between S₁ and T₁ must be less than 100 meV: 100 meV. This value is often debated, as it depends on the fluorophore studied: certain molecules having a ΔE_{ST} greater than 100 meV can, all the same, have delayed fluorescence properties. In literature, the upper limit of 360 meV is also found. ΔE_{ST} is directly proportional to the orbital overlapping between the HOMO and the LUMO of a molecule. Thus, the greater the separation between these two orbitals will be, the lower the ΔE_{ST} will be. The challenge of a molecular design to have a significant intersystem crossing resides in the obtaining of a good spatial separation of these frontier orbitals. Various molecular designs have been developed to fulfil these conditions, the most commonly used bringing into play donor-acceptor-type fluorophores, or these two units form a dihedral angle as close as possible to 90°. This makes it possible to position the HOMO mainly on the donor group and the LUMO mainly on the acceptor. However, it must be ensured to not totally separate the two frontier orbitals, as the quantum efficiency is proportional to the orbital overlapping between the HOMO and the LUMO. A compromise must therefore be found between a good separation to minimise ΔE_{ST} and to preserve interesting quantum efficiencies.

Thermally Activated Delayed Fluorescence

After the reverse intersystem crossing, the molecule returns to the state S₁.

Subsequently, it is possible for it to emit the fluorescence (deexcitation S₁→S₀) which will have the same emission wavelength as prompt fluorescence. However, as the molecule is moved through several states, the lifetime of this fluorescence is different, it is longer, of around 10⁻⁸ to 10 seconds, that is why this phenomenon is called “delayed fluorescence”. However, the term “delayed fluorescence” omits a significant part of the phenomenon. The full name for it is “thermally activated delayed fluorescence” (TADF). Due to the reverse intersystem crossing, which is a temperature-dependent process, the delayed fluorescence is also dependent on the temperature. Thus, the more the temperature of the medium increases, the more the intersystem crossing will be favoured, and the more the molecules can return to the state S₁ to emit the delayed fluorescence.

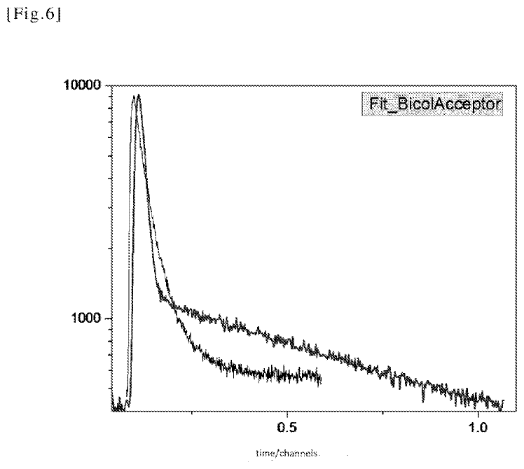

FIG. 4 shows the different phenomena present after excitation with their respective lifetime. The three radiative deexcitation processes presented, namely, the prompt fluorescence, the phosphorescence and the delayed fluorescence, are manifestations of luminescence. When the excitation is luminous, this is photoluminescence.

Photophysical Data and TADF:

To demonstrate the TADF properties of the compounds of the invention, it must be
verified that the fluorescence decay is biexponential: short lifetime of around one nanosecond and a long lifetime in degassed solution (oxygen of the air quenches, the triplet state, and
seen if an increase of the quantum efficiency (ϕ_{F}) is observed between the solution in air and the degassed solution in argon.

### Example 1: Synthesis of B2-CNPyrF₂

Chemical formula: C₃₄H₂₂F₂N₄O₂

Molar mass: 556.57 g·mol⁻¹

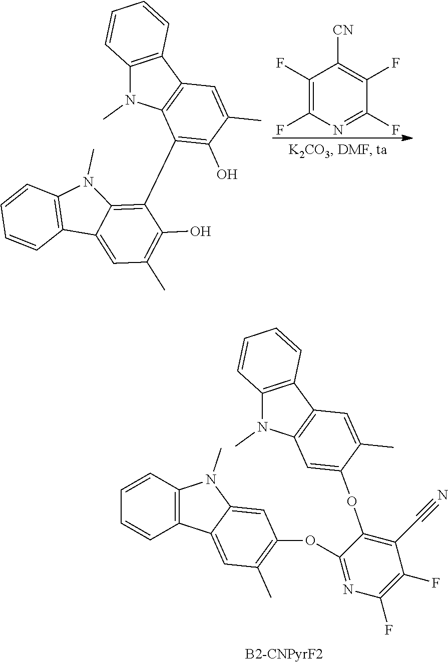

TABLE 1

| | Molar | | | Number of | Number |
|---|---|---|---|---|---|
| | mass | | | moles | of |
| Reagents | (gmol⁻¹) | Quantity | | (mmol) | equivalents |
| Bicol B2 | 420.51 | 0.0310 | g | 0.07 | 1 |
| Tetrafluoro- | 176.07 | 0.0167 | g | 0.09 | 1.3 |
| cyanopyridine | | | | | |
| CNPyrF₄ | | | | | |
| K₂CO₃ | 138.21 | 0.0376 | g | 0.27 | 3 |
| DMF | — | 1.0 | mL | — | — |

In a flask containing 3,3′,9,9′-tetramethyl-9H,9′H-[1,1′-bicarbozole]-2,2′-diol (B2, 0.0310 g, 0.07 mmol), 2,3,5,6-tetrafluoro-4-pyridinecarbonitrile (CNPyrF₄, 0.0167 g, 0.09 mmol) and K₂CO₃ (0.0376 g, 0.27 mmol), DMF (1 mL) is added under nitrogen atmosphere. The reactional mixture is stirred at ambient temperature (20±5° C.) for 16 hours. The reaction is stopped by adding distilled water and dichloromethane to the pale yellow suspension formed. The mixture is extracted twice with dichloromethane. The combined organic phases are dried on MgSO₄, filtered and concentrated under reduced pressure to give a purified yellow powder by flash chromatography on silica gel by using a cyclohexane/dichloromethane (1/1) mixture as eluent.

5,6-difluoro-2,9,15,16-tetramethyl-15,16-dihydropyrido[2′,3′:2,3] [1,4]dioxocino[6,5-a:7,8-a′] dicarbazole-7-carbonitrile (B2-CNPyrF₂) is obtained in the form of a white solid (0.0470 g, efficiency (calculated with respect to the starting BICOL B2)=99%) and characterised by RMN. The two enantiomers are then separated by chiral chromatography in supercritical phase.

RMN¹H (CDCl₃, 400 MHz): θ (ppm)=149.19, 148.50, 142.89, 142.79, 126.39, 126.35, 123.09, 123.04, 122.33, 122.15, 122.10, 121.41, 120.31, 120.29, 119.96, 119.93, 112.02, 111.84, 109.19, 109.17, 30.66, 30.59, 17.91, 17.26.

Quantum Efficiencies Measured in Toluene:

ϕ_{F} (O₂)=5% (prompt fluo); ϕ_{F} (Ar)=11% (prompt fluo+delayed fluo).

“Prompt fluo” means prompt fluorescence, i.e. short-lifetime fluorescence, of around ten ns, corresponding to the radiative deexcitation of the state S₁ to the fundamental state without moving through the triplet state;

“Delayed fluo” means delayed fluorescence, i.e. radiative deexcitation coming from relaxation from the low-energy excited singlet preceded by an intersystem crossing (reverse intersystem) between the low-energy singlet and triplet states.

The equipment and the method used to measure these parameters have already been specified.

To determine the quantum efficiency, the fluorescence signal of the product obtained is compared to that of a known ϕ_{F} reference, emitting in a close wavelength range. In the present case, said known ϕ_{F} reference is coumarin 102.

g_{light} for this compound is 0.8×10⁻³ (toluene=C=10⁻⁵).

### Example 2: Synthesis of B2-TPNF₂

Chemical formula: C₃₆H₂₂F₂N₄O₂

Molar mass: 580.59 g·mol⁻¹

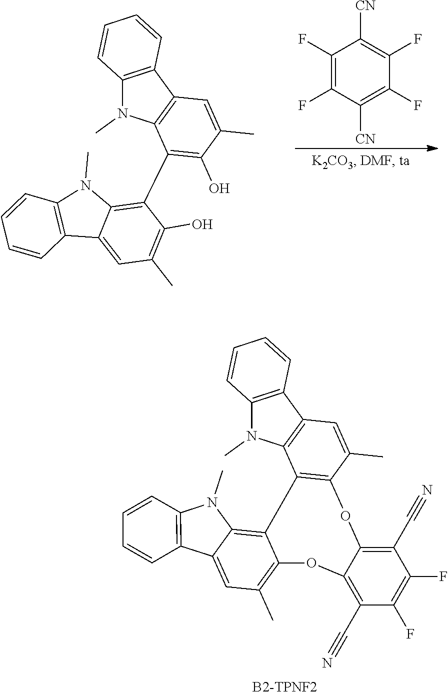 [SMILES: Cc1cc2c3ccccc3n(C)c2c(-c2c(O)c(C)cc3c4ccccc4n(C)c23)c1O ; N#Cc1c(F)c(F)c(C#N)c(F)c1F ; Cc1cc2c3ccccc3n(C)c2c2c1oc1c(C#N)c(F)c(F)c(C#N)c1oc1c(C)cc3c4ccccc4n(C)c3c12]

Molar mass: 420.51

TABLE 2

| | | | | Number | |
|---|---|---|---|---|---|
| | | | | of | Number |
| | MM | | | moles | of |
| Reagents | (gmol⁻¹) | Quantity | | (mmol) | equivalents |
| Bicol B2 | 420.51 | 0.0500 | g | 0.12 | 1 |
| Tetrafluoro- | 200.09 | 0.0287 | g | 0.14 | 1.2 |
| terephthalo- | | | | | |
| nitrile TPNF₄ | | | | | |
| K₂CO₃ | 138.21 | 0.0551 | g | 0.40 | 3 |
| DMF | — | 1.5 | mL | — | — |

In a flask containing BicolB (0.0500 g, 0.12 mmol), tetrafluoroterephtalonitrile (0.0287 g, 0.14 mmol) and K₂CO₃ (0.0551 g, 0.40 mmol), DMF (1.5 mL) is added under nitrogen atmosphere. The reactional mixture is stirred at ambient temperature (20±5° C.) for 12 hours. The reaction is stopped by adding distilled water and dichloromethane to the pale yellow suspension formed. The mixture is extracted twice with dichloromethane. The combined organic phases are dried on MgSO₄, filtered and concentrated under reduced pressure to give a purified yellow powder by flash chromatography on silica gel by using a cyclohexane/dichloromethane (1/1) mixture as eluent.

The compound B2-TPNF₂ is obtained in the form of a yellow solid (0.0455 g, efficiency (calculated with respect to the starting BICOL B2)=65%). The two enantiomers are then separated by chiral chromatography in supercritical phase.

RMN¹H (CDCl₃, 400 MHz): θ (ppm)=8.03 (d, 7.50 Hz, 2H), 8.00 (s, 2H), 7.41 (dd, 11.45 Hz, 3.97 Hz, 2H), 7.23 (d, 4H), 7.17, 3.12 (s, 6H), 2.52 (s, 6H)

RMN¹³C (CDCl₃, 100 MHz): θ (ppm)=149.49, 142.91, 138.10, 126.54, 123.35, 122.44, 122.03, 121.23, 120.36, 120.06, 111.90, 109.24, 29.86, 18.04.

Quantum Efficiencies Measured in Toluene:

ϕ_{F} (O₂)=10% (prompt fluo); ϕ_{F} (Ar)=26% (prompt fluo+delayed fluo).

g_{light} measured for this compound is 1.8×10⁻³ (toluene=C=10⁻⁵).

### Example 3: Synthesis of B1-TPNF₂

Chemical formula: C₃₄H₁₈F₂N₄O₂

Molar mass: 552.54 g·mol⁻¹

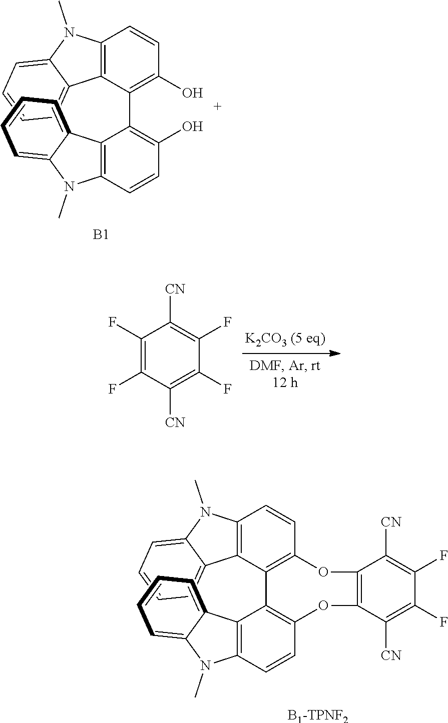

TABLE 3

| | | | | Number of | Number |
|---|---|---|---|---|---|
| | Molar mass | | | moles | of |
| Reagents | (gmol⁻¹) | Quantity | | (mmol) | equivalents |
| Bicol B1 | 392.46 | 0.0400 | g | 0.13 | 1 |
| Tetrafluoro- | 200.09 | 0.0220 | g | 0.13 | 1 |
| terephthalo- | | | | | |
| nitrile TPNF₄ | | | | | |
| K₂CO₃ | 138.21 | 0.0700 | g | 0.63 | 5 |
| DMF | — | 5.0 | mL | — | — |

In a flask containing 9,9′-tetramethyl-9H,9′H-[4,4′-bicarbazole]-3,3′-diol (B1, 0.040 g, 0.13 mmol), tetrafluoroterephthalonitrile (TPNF₄, 0.022 g, 0.13 mmol) and K₂CO₃ (0.070 g, 0.63 mmol), DMF (5 mL) is added under argon atmosphere. The reactional mixture is stirred at ambient temperature (20±5° C.) for 16 hours. The reaction is stopped by adding distilled water and ethyl acetate to the pale yellow suspension formed. The mixture is extracted twice with ethyl acetate. The combined organic phases are dried on MgSO₄, filtered and concentrated under reduced pressure to give a purified yellow powder by flash chromatography on silica gel by using a cyclohexane/ethyl acetate (1/1) mixture as eluent.

5,6-difluoro-2,9,15,16-tetramethyl-15,16-dihydropyrido[2′,3′:2,3] [1,4]dioxocino[6,5-a:7,8-a′] dicarbazole-7-carbonitrile (B1-TPNF₂) is obtained in the form of a white solid (0.0430 g, efficiency (calculated with respect to the starting BICOL)=76%). The two enantiomers are then separated by chiral chromatography in supercritical phase.

RMN¹H (CD2Cl₂, 400 MHz): θ (ppm)=7.64 (d, J=8.8 Hz, 2H), 7.56 (d, J=8.8 Hz, 2H), 7.45 (d, J=8.3 Hz, 2H), 7.34 (ddd, J=8.3, 7.1, 1.2 Hz, 2H), 6.94 (d, J=8.0 Hz, 2H), 6.64 (ddd, J=8.0, 7.0, 1.0 Hz, 2H), 3.98 (s, 6H).

RMN¹³C (CD2Cl₂, 100 MHz): θ (ppm)=148.5, 147.8, 145.3, 145.2, 144.7, 142.2, 139.4, 126.5, 122.8, 122.0, 121.6, 120.9, 118.9, 118.6, 110.0, 109.1, 108.7, 102.8, 29.4.

HRMS calculated for [M+Na]⁺ (C₃₄H₁₈N₄O₂F₂Na): 575.129 found 575.1288 (0 ppm).

Quantum Efficiencies Measured in Toluene:

ϕ_{F} (O₂)=6% (prompt fluo); ϕ_{F} (Ar)=15% (prompt fluo+delayed fluo).

g_{light} measured for this compound is 0.7×10⁻³ (toluene=C=10⁻⁵).

### Example 4: Synthesis of C1-(S)-TPNBINOL-(R)

Chemical formula: C₆₀H₃₄N₄O₂

Molar mass: 842.2682 g·mol⁻¹

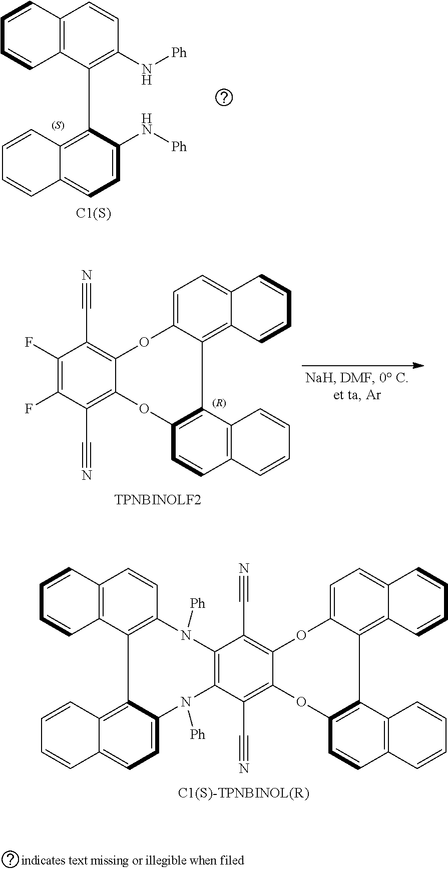

TABLE 4

| | | | | Number | |
|---|---|---|---|---|---|
| | Molar | | | of | Number |
| | mass | | | moles | of |
| Reagents | (gmol⁻¹) | Quantity | | (mmol) | equivalents |
| (S)-N2,N2′- | 436.19 | 0.060 | g | 0.14 | 1 |
| diphenyl-[1,1′- | | | | | |
| binaphthalane]- | | | | | |
| 2,2′-diamine | | | | | |
| (C1) | | | | | |
| TPNBINOLF₂ | 446.08 | 0.061 | g | 0.14 | 1 |
| NaH (60% in | 24 | 0.012 | g | 0.30 | 2.2 |
| oil) | | | | | |
| DMF | — | 2 | mL | — | — |

In a flask containing N2,N2′-diphenyl-[1,1′-binaphthalane]-2,2′-diamine (C1(S), 0.060 g, 0.14 mmol) solubilised in DMF (1 mL), NaH (0.012 g, 0.30 mmol) is added under argon atmosphere at 0° C. The reactional mixture is stirred at 0° C. for 5 minutes, then the reactional mixture is left to return to ambient temperature (about 10 minutes). Then, a solution of the compound TPNBINOLF₂ (0.061 g, 0.14 mmol) in DMF (1 mL) is slowly added to the reactional mixture. After this addition, the solution is stirred for a night at ambient temperature. The reaction is stopped by adding distilled water and ethyl acetate. The mixture is extracted twice with ethyl acetate. The combined organic phases are dried on MgSO₄, filtered and concentrated under reduced pressure to give a purified yellow powder by flash chromatography on silica gel by using a cyclohexane/ethyl acetate (80/20) mixture as eluent.

The product C1-(S)-TPNBINOL-(R) is obtained in the form of a yellow solid (0.0740 g, efficiency (calculated with respect to the starting diamine)=64%) and characterised by RMN.

RMN¹H (400 MHz, CDCl₃) 08.13 (d, J=8.8 Hz, 2H), 8.01 (d, J=8.2 Hz, 2H), 7.95 (d, J=8.5 Hz, 2H), 7.79 (d, J=8.8 Hz, 2H), 7.75 (d, J=8.5 Hz, 2H), 7.71 (d, J=8.3 Hz, 2H), 7.56-7.52 (m, 2H), 7.47 (d, J=8.0 Hz, 2H), 7.43-7.37 (m, 2H), 7.32-7.38 (m, 2H), 7.14-7.06 (m, 4H), 6.59 (t, J=7.4 Hz, 2H), 6.45, (d, J=8.1 Hz, 2H), 6.31 (t, J=7.4 Hz, 2H), 6.21 (dd, J=7.8, 6.8 Hz, 2H), 5.48 (d, J=8.0 Hz, 2H).

RMN¹³C (101 MHz, CDCl₃) 0149.7, 148.6, 141.4, 139.6, 137.0, 132.8, 132.2, 132.2, 132.0, 131.3, 130.6, 128.4, 128.3, 127.7, 127.6, 127.4, 127.3, 126.6, 126.3, 126.2, 126.0, 125.9, 125.0, 121.0, 120.7, 118.4, 114.2, 112.8, 112.3.

HRMS calculated for [M+Na]⁺ (C₃₄H₁₈N₄O₂F₂Na): 843.2760 found 843.2760 (0 ppm).

Quantum Efficiencies Measured in Toluene:

ϕ_{F} (O₂)=5% (prompt fluo); ϕ_{F} (Ar)=9% (prompt fluo+delayed fluo).

g_{light} measured for this compound is 1.6×10⁻³ (toluene=C=10⁻⁵).

### Example 5: Comparative Example

The molecule of formula A1

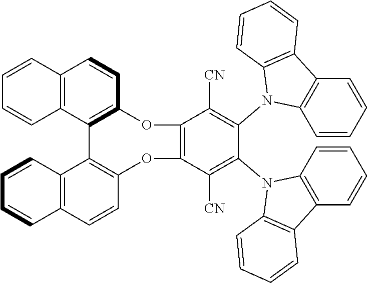

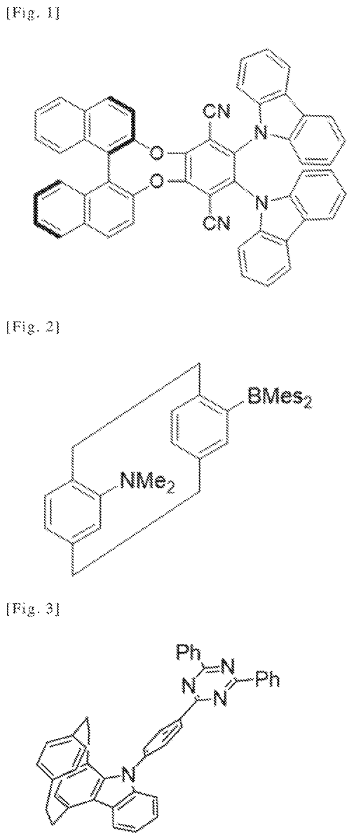

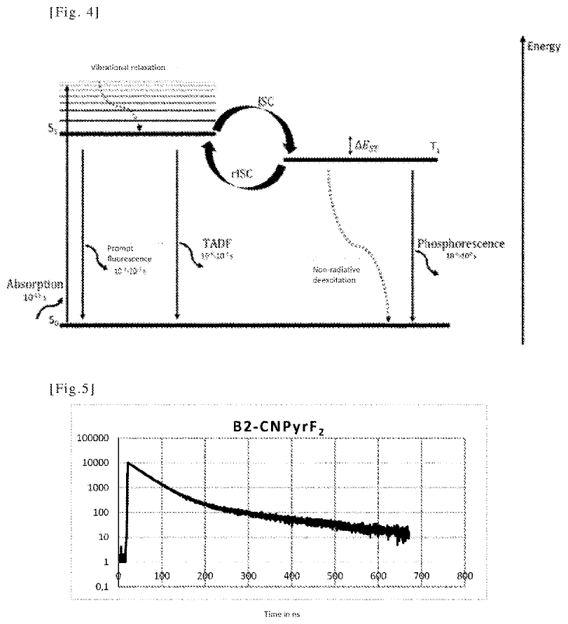

represented in [FIG. 1] and described by G. Pieters et al. (J. Am. Chem. Soc. 2016, 138, 3990-3993) has been synthetised according to the operating mode described in this publication.

The CPL data of molecule A1 and compounds B2-CNPyrF₂ of example 1 and B2-TPNF₂ of example 2 have been calculated according to the method described above and compared.

Molecule A1: g_{light} (λₘₐₓ)=0.3×10⁻³

B2-CNPyrF₂: g_{light} (λₘₐₓ)=0.8×10⁻³

B2-TPNF₂: g_{light} (λₘₐₓ)=1.8×10⁻³

B1-TPNF₂: g_{light} (λₘₐₓ)=0.7×10⁻³

C1-TPNBinol: g_{light} (λₘₐₓ)=1.6×10⁻³

In conclusion, with respect to the CPTADF molecule (molecule A1) having AIEE properties, the compounds B2-TPNF₂, B2-CNPyrF₂, B1-TPNF₂ and C1(S)-TPNBINOL(R) have dissymmetry factor values (glum) that is greater (up to 6 times).

1. A compound of formula (I) 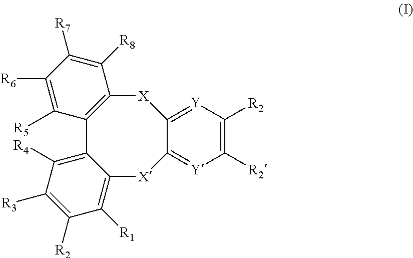
wherein
X and X′, identical or different, represent a heteroatom chosen from the group formed by O, and N—R₉ with R₉ representing a hydrogen atom, a deuterium, an alkyl radical comprising 1 to 12 carbon atoms or an aryl radical comprising 6 to 20 carbon atoms, said alkyl and aryl radicals being optionally substituted; and
Y and Y′, identical or different, represent C—R_{y}, C—R_{y′}, a heteroatom chosen from the group formed by N, O, with R_{y} and R_{y′}, identical or different, representing a hydrogen atom, a deuterium, a nitrile group (—CN), a halogen atom chosen from the group formed by F, Cl, Br and I, an alkyl radical comprising 1 to 12 carbon atoms, an aryl radical comprising 6 to 20 carbon atoms, a hydroxyl group, an alkoxy group, an aryloxy group, an amine group of formula NR₁₄R₁₅ with R₁₄ and R₁₅, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted; and
R_{z} and R_{z′}, identical or different, represent a hydrogen atom, a deuterium, a nitrile group (—CN), a halogen atom chosen from the group formed by F, Cl, Br and I, an alkyl radical comprising 1 to 12 carbon atoms, an aryl radical comprising 6 to 20 carbon atoms, a hydroxyl group, an alkoxy group, an aryloxy group, an amine group of formula NR₁₇R₁₈ with R₁₇ and R₁₈, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted;
or
R_{z} and R_{z′}, together with the carbon atoms to which they are bonded, form an aryl or a heterocycle chosen from the group formed by: 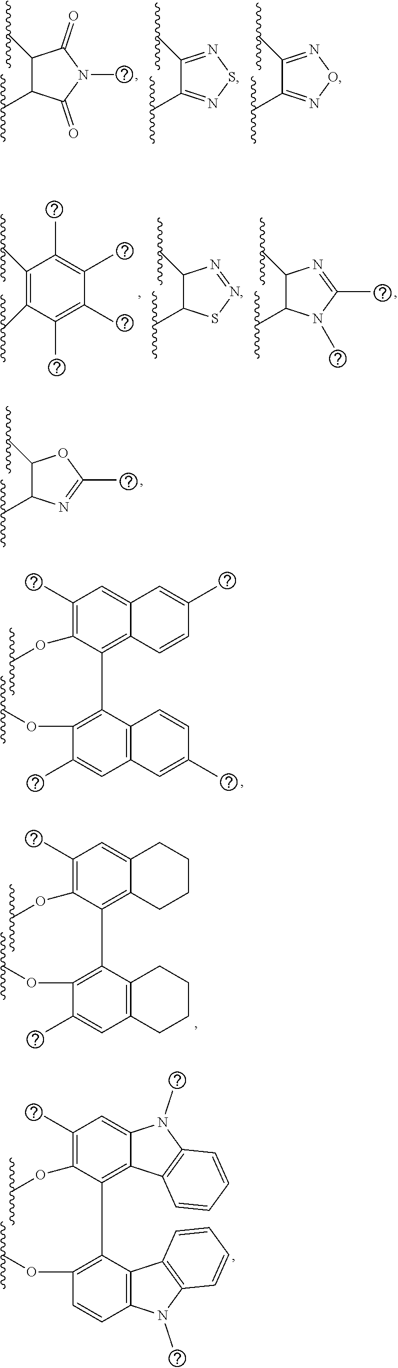 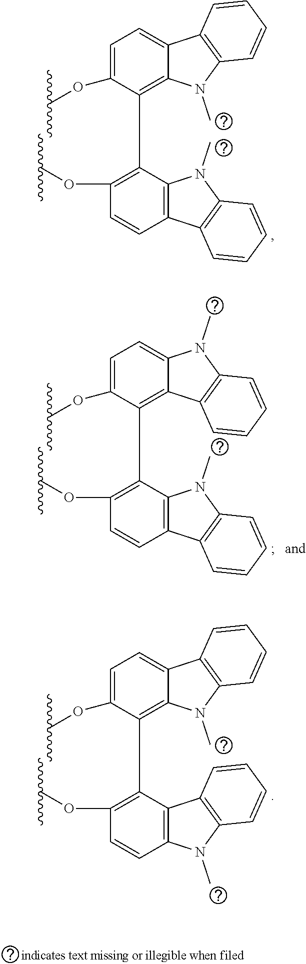 [SMILES: *Oc1ccc2c3ccccc3n(C)c2c1-c1c(O*)ccc2c3ccccc3n(C)c12 ; *Oc1ccc2c(c1-c1c(O*)ccc3c4ccccc4n(C)c13)c1ccccc1n2C ; *Oc1ccc2c(c1-c1c(O*)ccc3c4ccccc4n(C)c13)c1ccccc1n2C]
with R₁₀, R₂₀, R₂₁, R₂₂, R₂₃, R₂₄, R₂₅, R₂₆, R₂₇, R₂₈, R₂₉, R₃₀, R₃₁, R₃₂, R₃₃ and R₃₄, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted;
R₁, R₂, R₃, R₄, R₅, R₆, R₇ and R₈, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₁₂R₁₃ with R₁₂ and R₁₃, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted;
or
R₁, R₂, R₇ and R₈, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₄₀R₄₁ with R₄₀ and R₄₁, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted, and
R₃ and R₄, together with the carbon atoms to which they are bonded, form a cyclic alkyl, an aryl or a heterocycle chosen from the group formed by: 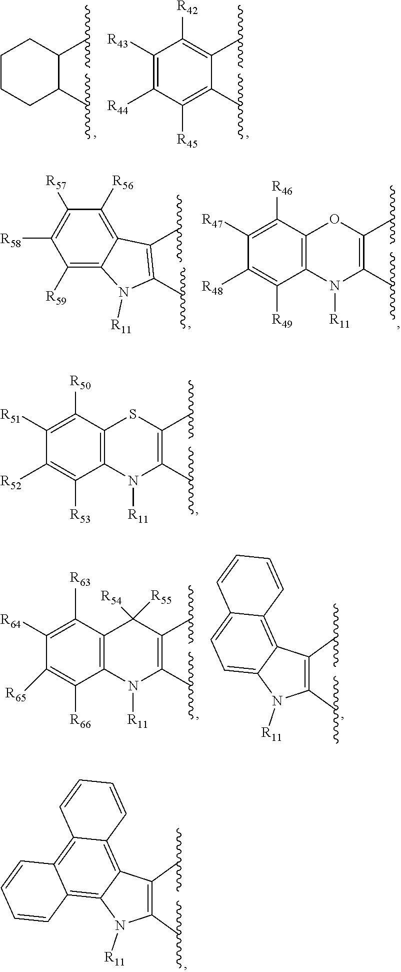
and
R₅ and R₆, together with the carbon atoms to which they are bonded, form a cyclic alkyl, an aryl or a heterocycle chosen from the group formed by: 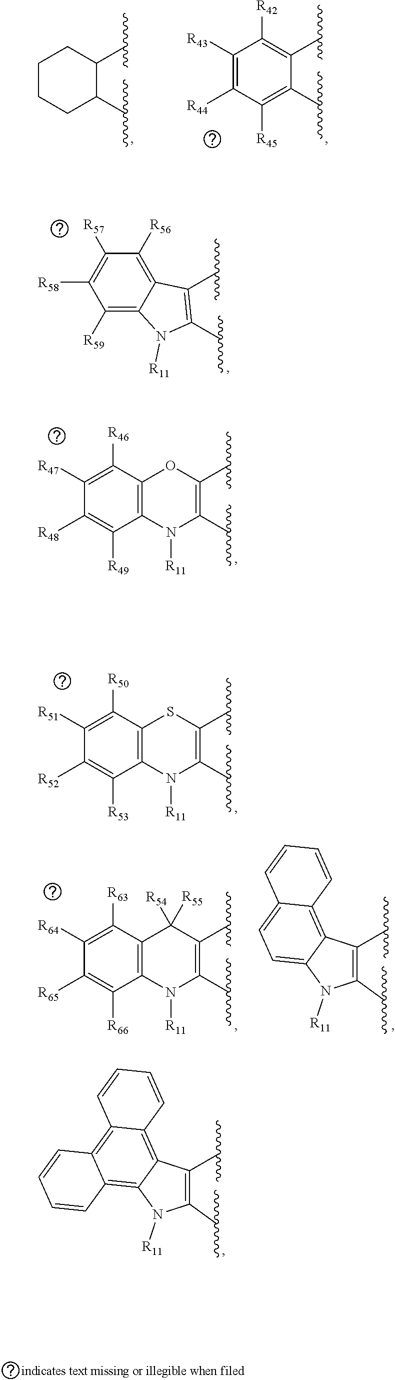
with R₁₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₃, R₆₄, R₆₅ and R₆₆, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₆₁R₆₂ with R₆₁ and R₆₂, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted;
or
R₁ and R₂, on the one hand, and R₇ and R₈, on the other hand, together with the carbon atoms to which they are bonded, each a naphthyl to lead to a fragment of the following formula: 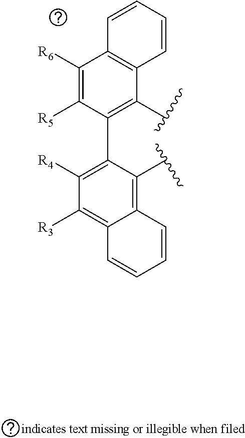
with
R₃, R₄, R₅ and R₆, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₁₂R₁₃ with R₁₂ and R₁₃, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted; or
R₃ and R₄, together with the carbon atoms to which they are bonded, form a cyclic alkyl, an aryl or a heterocycle chosen from the group formed by: 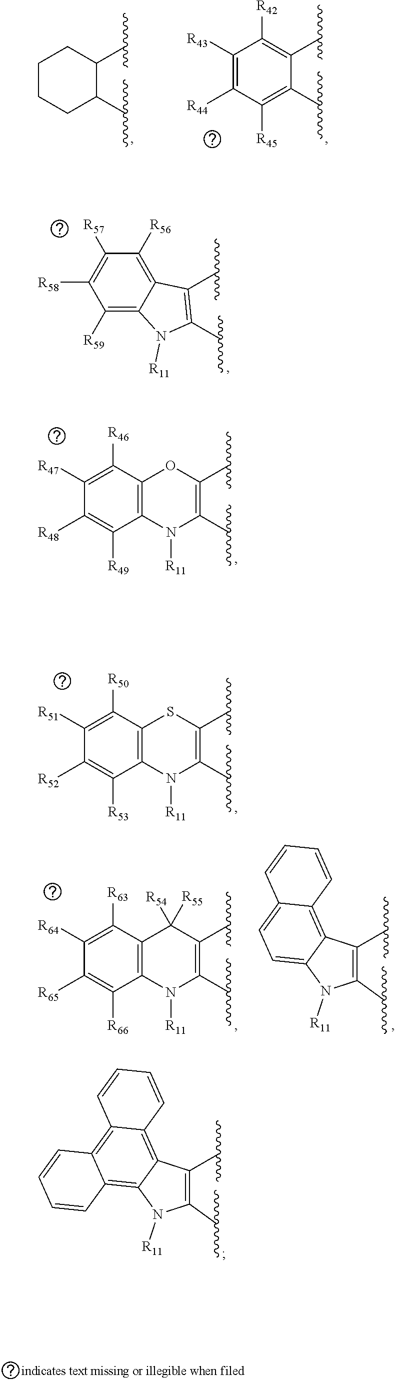
and
R₅ and R₆, together with the carbon atoms to which they are bonded, form a cyclic alkyl, an aryl or a heterocycle chosen from the group formed by: 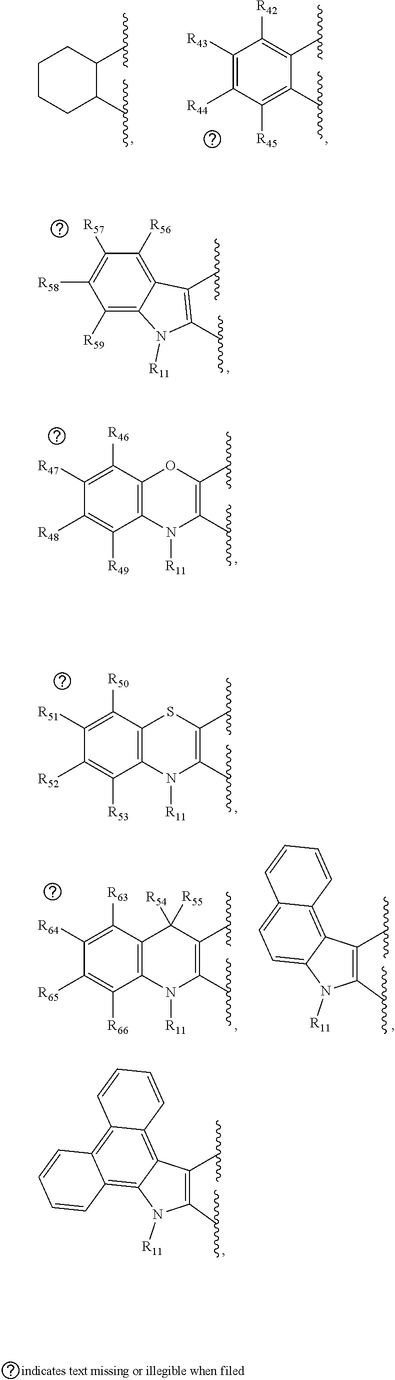
with R₁₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₃, R₆₄, R₆₅ and R₆₆, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₆₁R₆₂ with R₆₁ and R₆₂, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted;
excluding the following compounds

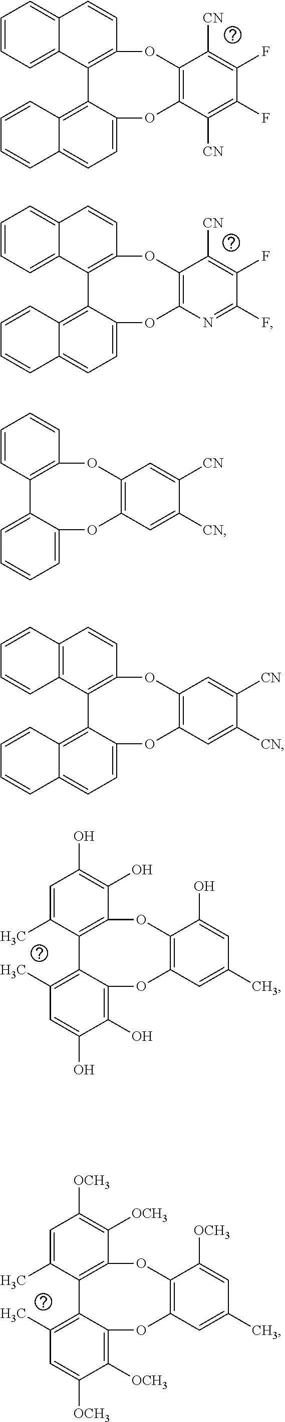

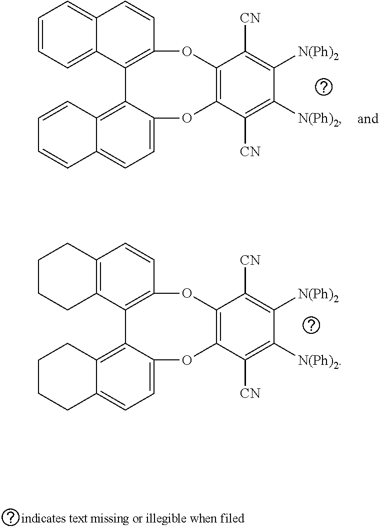

2. The compound according to claim 1, wherein
Y and Y′ represent C—R_{y}, C—R_{y′} with R_{y} and R_{y′}, representing a nitrile group (—CN), and R_{z} and R_{z′}, identical or different, represent a halogen atom chosen from the group formed by F and Cl; or
Y and Y′ represent C—R_{y}, C—R_{y′} with R_{y} and R_{y′}, identical or different, representing a halogen atom chosen from the group formed by F and C1, and R_{z} and R_{z′}, represent a nitrile group (—CN);
or
Y and Y′ represent C—R_{y}, C—R_{y′} with R_{y} and R_{y′}, identical or different, representing a hydrogen atom, a deuterium, an aryl radical comprising 6 to 20 carbon atoms, said aryl radical being optionally substituted, and R_{z} and R_{z′}, represent a nitrile group (—CN).

3. The compound according to claim 1, wherein
Y and Y′, identical or different, represent C—R_{y}, C—R_{y′}, a heteroatom chosen from the group formed by N and O, with R_{y} and R_{y′}, identical or different, being a hydrogen atom, a deuterium, a nitrile group (—CN), a halogen atom chosen from the group formed by F and Cl, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms, said alkyl and aryl radicals being optionally substituted; and
R_{z} and R_{z′}, together with the carbon atoms to which they are bonded, form: 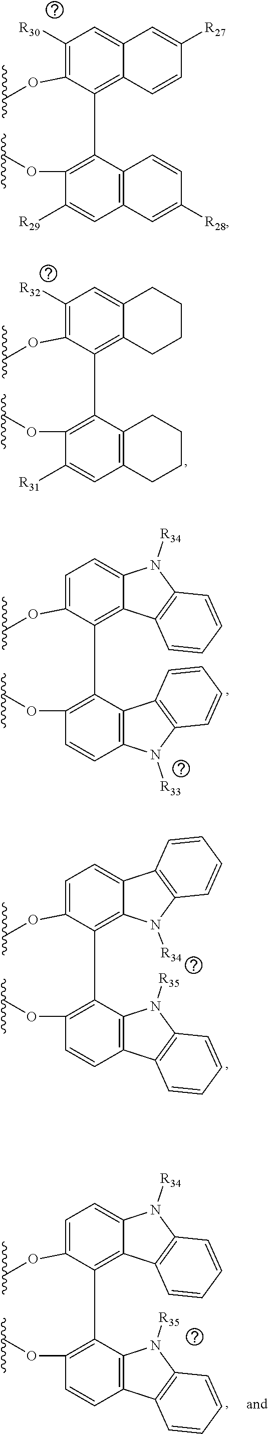 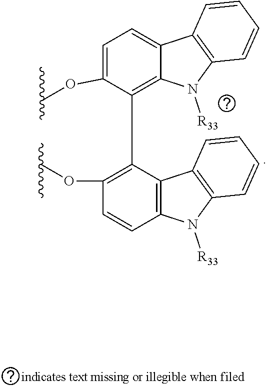
with R₂₇, R₂₈, R₂₉, R₃₀, R₃₁, R₃₂, R₃₃ and R₃₄, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted.

4. The compound according to claim 1, wherein
Y and Y′, identical or different, represent a heteroatom chosen from the group formed by N and O, C—R_{y}, C(R_{y′}) with R_{y} and R_{y′}, identical or different, being a hydrogen atom, a deuterium, a nitrile group (—CN), a halogen atom chosen from the group formed by F and Cl, an alkyl radical comprising 1 to 12 carbon atoms, an aryl radical comprising 6 to 20 carbon atoms, an amine group of formula NR₃₅R₃₆ with R₃₅ and R₃₆, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted; and
R_{z} and R_{z′}, together with the carbon atoms to which they are bonded, form a heterocycle chosen from the group formed by: 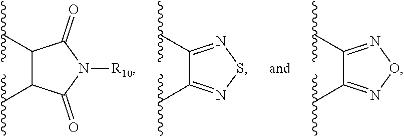
with R₁₀ representing a hydrogen atom, a deuterium, an alkyl radical comprising 1 to 12 carbon atoms, an aryl radical comprising 6 to 20 carbon atoms,
said alkyl and aryl radicals being optionally substituted.

5. The compound according to claim 1, wherein
Y and Y′, identical or different, represent a heteroatom chosen from the group formed by N and O, C—R_{y}, C—R_{y} with R_{y} and R_{y′}, identical or different, representing a hydrogen atom, a deuterium, a nitrile group (—CN); and
R_{z} and R_{z′}, identical or different, represent a hydrogen atom, a deuterium, a nitrile group (—CN), a halogen atom chosen from the group formed by F and Cl.

6. The compound according to claim 1, wherein
Y and Y′ represent N and R_{z} and R_{z′} together with the carbon atoms to which they are bonded, form: 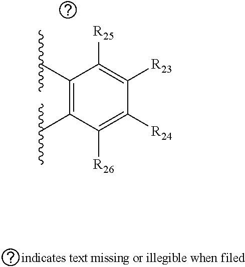
with R₂₃, R₂₄, R₂₅ and R₂₆, identical or different, representing a hydrogen atom, a deuterium, an alkyl radical comprising 1 to 12 carbon atoms, an aryl radical comprising 6 to 20 carbon atoms,
said alkyl and aryl radicals being optionally substituted.

7. The compound according to claim 1, wherein
X and X′, represent O; and
R₁, R₂, R₃, R₄, R₅, R₆, R₇ and R₈, identical or different, represent a hydrogen atom, a deuterium, an alkyl radical comprising 1 to 12 carbon atoms, an amine group of formula NR₁₂R₁₃ with R₁₂ and R₁₃, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted.

8. The compound according to claim 1, wherein
X and X′, identical or different, represent a heteroatom chosen from the group formed by O, and N—R₉ with R₉ representing a hydrogen atom, a deuterium, an alkyl radical comprising 1 to 12 carbon atoms or an aryl radical comprising 6 to 20 carbon atoms, said alkyl and aryl radicals being optionally substituted; and
R₁, R₂, R₇ and R₈, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₄₀R₄₁ with R₄₀ and R₄₁, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted, and
R₃ and R₄, together with the carbon atoms to which they are bonded, form a cyclic alkyl or an aryl chosen from the group formed by: 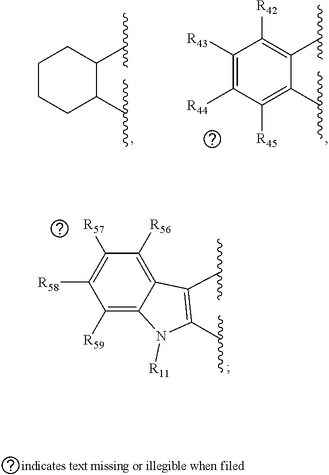
and
R₅ and R₆, together with the carbon atoms to which they are bonded, form a cyclic alkyl or an aryl chosen from the group formed by: 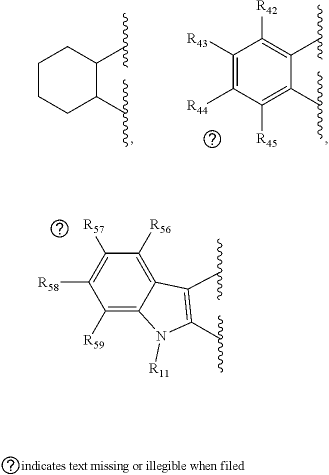
with
R₁₁, R₄₂, R₄₃, R₄₄, R₄₅, R₅₆, R₅₇, R₅₈ and R₅₉, identical or different, represent a hydrogen atom, a deuterium, an alkyl radical comprising 1 to 12 carbon atoms, an aryl radical comprising 6 to 20 carbon atoms, a hydroxyl group, an alkoxy group, an aryloxy group, an amine group of formula NR₆₁R₆₂ with R₆₁ and R₆₂, identical or different, representing a hydrogen atom, a deuterium, an alkyl radical comprising 1 to 12 carbon atoms or an aryl radical comprising 6 to 20 carbon atoms,
said alkyl and aryl radicals being optionally substituted.

9. The compound according to claim 1, wherein
X and X′, identical or different, represent a heteroatom chosen from the group formed by O, and N—R₉ with R₉ represent a hydrogen atom, a deuterium, an alkyl radical comprising 1 to 12 carbon atoms or an aryl radical comprising 6 to 20 carbon atoms, said alkyl and aryl radicals being optionally substituted;
R₁, R₂, R₇ and R₈, identical or different, represent a hydrogen atom, a deuterium, an alkyl radical comprising 1 to 12 carbon atoms, an aryl radical comprising 6 to 20 carbon atoms, said alkyl and aryl radicals being optionally substituted, and
R₃ and R₄, together with the carbon atoms to which they are bonded, form a cyclic alkyl or an aryl chosen from the group formed by: 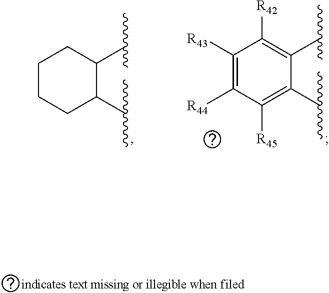
and
R₅ and R₆, together with the carbon atoms to which they are bonded, form a cyclic alkyl or an aryl chosen from the group formed by: 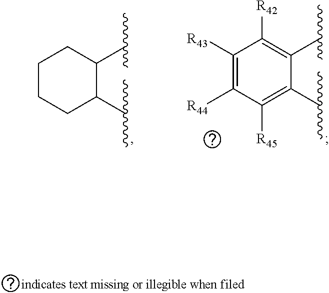
with R₄₂, R₄₃, R₄₄ and R₄₅, identical or different, representing a hydrogen atom, a deuterium, an alkyl radical comprising 1 to 12 carbon atoms, an aryl radical comprising 6 to 20 carbon atoms,
said alkyl and aryl radicals being optionally substituted.

10. The compound of formula (I) according to claim 1,
wherein
X and X′, identical or different, represent a heteroatom chosen from the group formed by O and N—R₉ with R₉ representing a hydrogen atom, a deuterium, an alkyl radical comprising 1 to 12 carbon atoms or an aryl radical comprising 6 to 20 carbon atoms, said alkyl and aryl radicals being optionally substituted; and
R₁, R₂, R₇ and R₈, identical or different, represent a hydrogen atom, a deuterium, an alkyl radical comprising 1 to 12 carbon atoms, an aryl radical comprising 6 to 20 carbon atoms,
said alkyl and aryl radicals being optionally substituted as indicated above, and
R₃ and R₄, together with the carbon atoms to which they are bonded, form: 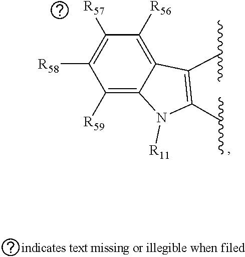
and
R₅ and R₆, together with the carbon atoms to which they are bonded, form: 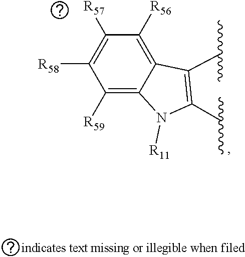
with
R₁₁, R₅₆, R₅₇, R₅₈ and R₅₉, identical or different, representing a hydrogen atom, a deuterium, an alkyl radical comprising 1 to 12 carbon atoms, an aryl radical comprising 6 to 20 carbon atoms, said alkyl and aryl radicals being optionally substituted.

11. The compound of formula (I) according to claim 1,
wherein it is chosen from the group formed by:

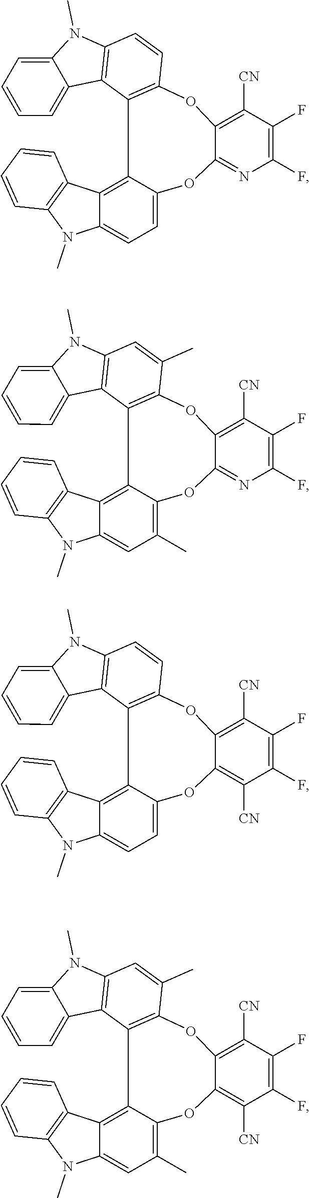

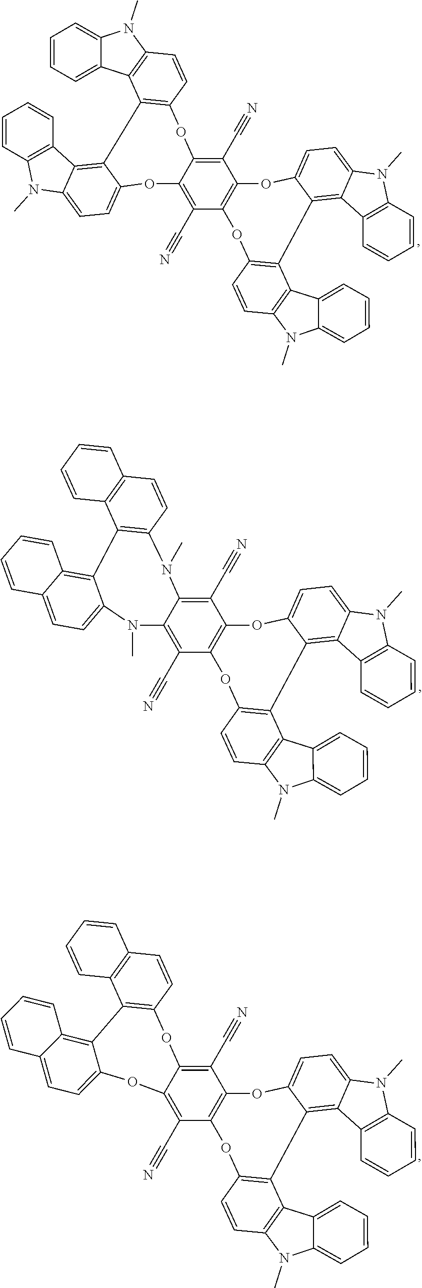 [SMILES: Cn1c2ccccc2c2c3c(ccc21)oc1c(C#N)c2oc4ccc5c(c6ccccc6n5C)c4c4c(ccc5c4c4ccccc4n5C)oc2c(C#N)c1oc1ccc2c(c4ccccc4n2C)c13 ; Cn1c2ccccc2c2c3c(ccc21)oc1c(C#N)c2c(c(C#N)c1oc1ccc4c(c5ccccc5n4C)c13)n(C)c1ccc3ccccc3c1c1c3ccccc3ccc1n2C ; Cn1c2ccccc2c2c3c(ccc21)oc1c(C#N)c2oc4ccc5ccccc5c4c4c(ccc5ccccc54)oc2c(C#N)c1oc1ccc2c(c4ccccc4n2C)c13]

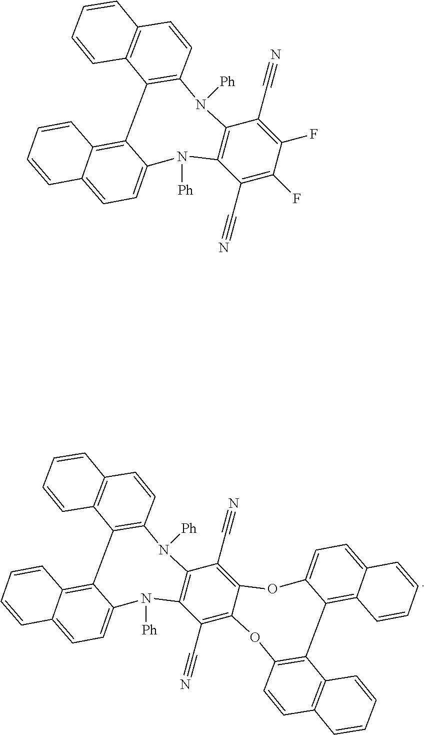 [SMILES: N#Cc1c(F)c(F)c(C#N)c2c1n(-c1ccccc1)c1ccc3ccccc3c1c1c3ccccc3ccc1n2-c1ccccc1 ; N#Cc1c2oc3ccc4ccccc4c3c3c(ccc4ccccc43)oc2c(C#N)c2c1n(-c1ccccc1)c1ccc3ccccc3c1c1c3ccccc3ccc1n2-c1ccccc1]

12. (canceled)

13. A light-emitting device or light-emitting diode (OLED) comprising a compound according to claim 1.
